(19) United States
(12) Patent Application Publication
(10) Pub. No.: US 2025/0108076 A1
(43) Pub. Date: Apr. 3, 2025

(54) METHODS FOR TREATMENT OF NON-ALCOHOLIC FATTY LIVER DISEASES (NAFLD) USING ADVANCED MICROBIOME THERAPEUTICS

(71) Applicants: Danmarks Tekniske Universitet, Kongens Lyngby (DK); UNIVERSITY OF EASTERN FINLAND, Kuopio (FI)

(72) Inventors: Morten Otto Alexander SOMMER, Kongens Lyngby (DK); Ruben Vazquyez URIBE, Kongens Lyngby (DK); Mareike BONGERS, Kongens Lyngby (DK); Hani EL-NEZAMY, Kongens Lyngby (DK); Marjukka KOLEHMAINEN, Kuopio (FI); Valeria IANNONE, Kuopio (FI); Johnson LOK, Kuopio (FI); Carlos Gomez GALLEGO, Kuopio (FI)

(21) Appl. No.: 18/857,837

(22) PCT Filed: Apr. 24, 2023
(86) PCT No.: PCT/EP2023/060599

§ 371 Date: Oct. 18, 2024

(30) Foreign Application Priority Data
Apr. 25, 2022 (EP) 22169704.8

(51) Int. Cl. A61K 35/744; A61K 38/18; A61K 38/30; A61P 1/16; C07K 14/50; C07K 14/65

(52) U.S. Cl. CPC A61K 35/744; A61K 38/1825; A61K 38/30; A61P 1/16; C07K 14/50; C07K 14/65

(57) ABSTRACT

The present invention relates to a composition comprising a genetically engineered non-pathogenic microorganism expressing one or more polypeptide comprising polypeptide hormones. The composition is preferably for use in the treatment of non-alcoholic fat liver disease (NAFLD).

### FIELD

The invention is in the field of metabolic disorders and complications from pathologies that result in unbalanced peptide hormone homeostasis. The invention relates to a composition comprising a system for secreting peptide hormones in the gastrointestinal tract, wherein the system comprises engineered non-pathogenic microorganism expressing a polypeptide comprising a polypeptide hormone. Specifically, the bioactive molecules are polypeptide hormones such FGF-19, Aldafermin and/or IGF1 and the non-pathogenic microorganism may be Escherichia coli, such as E. coli Nissle 1917 (EcN). The composition can be used for regulation of peptide hormone homeostasis and for prevention or treatment of disorders associated with unbalanced peptide hormone homeostasis. Such disorders include e.g., non-alcoholic fat liver disease, liver cirrhosis, cancer and hepatic inflammation. Thus, a composition of the invention can be used in the treatment of or in the avoidance or reduction in the development of NAFLD in the liver.

### BACKGROUND

Recent advances in bioinformatic technologies have elevated our understanding of the role the microbiome plays in host physiological processes including, but not limited to, metabolism, inflammation, behaviour and neurological diseases.

In Western countries, non-alcoholic fatty liver disease (NAFLD) is one of the most common chronic liver disease and multi-factorial disease. The pathogenesis and progression of NAFLD, from simple accumulation of fat in the liver to a more severe state (NASH) is poorly understood but it is explained by “multiple-hits theory”, in which genetic factors and diet can cause changes in the gastrointestinal (GI) tract regarding microbial composition and activity, insulin resistance, and obesity, which simultaneously disrupt metabolic activity of fat and liver. WO2018/195097 is an example of utilization of host endogenous microbiome species (native/commensal) that are isolated from the host, genetically engineered to express a heterologous polynucleotide, whereafter it is reintroduced into to the GI tract of host, to act as a therapeutic by expression and in some cases excreting said polypeptide locally into the GI tract, where it may exert its therapeutic function.

Due to the close metabolic relationship between the liver and the gastrointestinal tract, it is not surprising that intestinal microbial dysbiosis has been associated with all stages of non-alcoholic fatty liver disease (NAFLD) (See Compare, D., et al., Gut-liver axis: the impact of gut microbiota on non alcoholic fatty liver disease. Nutrition, Metabolism and Cardiovascular Diseases, 2012. 22(6): p. 471-476). Suggested mechanisms in the microbial regulation of NAFLD include alterations in microbiota composition and their derived metabolites translocating to the liver through a disrupted gut barrier, where they evoke a hepatic inflammatory reaction and steatosis (see Kolodziejczyk, A. A., et al., The role of the microbiome in NAFLD and NASH. EMBO molecular medicine, 2019. 11(2): p. e9302). Patients with NAFLD show increased intestinal permeability that correlates with the severity of steatosis (see Plaza-Diaz, J., et al., Safety and immunomodulatory effects of three probiotic strains isolated from the feces of breast-fed infants in healthy adults: SETOPROB study. PloS one, 2013. 8(10): p. e78111), indicating that disruption of the GI barrier function may be one of the key steps in the pathogenesis and progression of NAFLD. Qualitative or quantitative alterations in the microbiota can impair this symbiotic relationship, decreasing the GI tract barrier protection (see Rowland, I., et al., Gut microbiota functions: metabolism of nutrients and other food components. European journal of nutrition, 2018. 57(1): p. 1-24; Iacob, S. and D. G. Iacob, Infectious threats, the intestinal barrier, and its trojan horse: dysbiosis. Frontiers in microbiology, 2019. 10: p. 1676; and McDonald, D., et al., Extreme dysbiosis of the microbiome in critical illness. Msphere, 2016. 1(4)). Several metabolites have been shown to modulate gut barrier integrity. Endocrine unbalance and endocrine diseases are also commonly associated with NAFLD, and restoration of the hormonal homeostasis can contribute to inhibiting the cascade of metabolic events that lead the progression of the disease.

Firstline treatments of NAFDL is lifestyle modification therapies, and not all patients respond well and maintain the lifestyle modification, and relapse is very commonly seen. Thus, alternative treatment strategies are very welcomed by patients as well as practitioners. In recent years peptide hormones have gained impasse in the treatment of metabolic disorders such as obesity, which is a common precursor to NAFLD, and their use in the modulation of imbalances in peptide hormone homeostasis, in particular in the in the gastrointestinal (GI) tract.

### SUMMARY OF THE INVENTION

The object of the present invention is to provide a composition comprising genetically modified non-pathogenic microorganism capable of secreting a peptide hormone in the gastrointestinal (GI) tract, wherein the non-pathogenic microorganism expressing one or more polypeptides comprising polypeptide hormones. Preferably, the polypeptide hormones are selected from the group consisting of FGF-19, Aldafermin, IGF-1 and functional homologues thereof. Preferably, the composition is for use as a medicament, and more preferably, the composition is for use in the treatment of NAFLD. It is preferred that the non-pathogenic microorganism is E. coli, such as E. coli Nissle 1917 (EcN).

A first aspect of the invention relates to a composition comprising a non-pathogenic microorganism expressing one or more polypeptides, wherein said polypeptides comprise polypeptide hormones selected from the group consisting of FGF-19, Aldafermin, IGF-1 and functional homologues of FGF-19, Aldafermin and IGF-1, and wherein FGF-19 has an amino acid sequence identified by SEQ ID NO: 1, Aldafermin has an amino acid sequence identified by SEQ ID NO: 2 and IGF-1 has an amino acid sequence identified by SEQ ID NO: 3, and a functional homologue thereof has a sequence identity of at least 80%, such as 85%, such as 90%, such as 95% or such as at least 99% towards SEQ ID NO: 1, 2 or 3. Such polypeptides could be peptide hormones, fusion peptides, chimeric peptides, oligomeric peptides, peptide hormone variants, and/or combinations thereof. The expression and/or secretion of said polypeptides of the invention may be enhanced by an enhanced expression through addition of promoter elements, such as a Schazetta derived promoter, commonly placed upstream of the nucleic acid sequence encoding said polypeptide, or it may be enhanced through addition of an N-terminal signal peptide which, in the non-pathogenic microorganism signals for the secretion of the said polypeptides from the intracellular matrix into the extracellular matrix, such as the lumen of the intestines.

The invention also provides a nucleic acid construct encoding said polypeptides and a genetically engineered non-pathogenic microorganism expressing said polypeptides. The invention further provides compositions comprising combinations of the genetically engineered non-pathogenic microorganisms of the invention.

The secretion of a polypeptide comprising a polypeptide hormone of the present invention in the GI tract of a host may reduce the liver fat content of the host animal, and in that sense, it may reduce or ameliorate the fat liver disease state in non-alcoholic fat liver disease (NAFLD).

Accordingly, a second aspect of the invention relates to the use of the composition as a medicament. In that regard a composition of the invention can be used for modulation of imbalances in peptide hormone homeostasis in the gastrointestinal tract and for treatment or amelioration of obesity associated diseases, preferably NAFLD. Thus, the present invention also relates to a composition for use in the treatment of NAFLD.

The invention also provides a therapeutic regimen, wherein the dosage of the composition comprising the genetically engineered non-pathogenic microorganism may be determined on an individual basis, and the amount of the administrated microbiome-based therapeutic can be assessed and monitored on the basis of the individual patient's age, weight, food intake, macrobiotic flora, level of polypeptide hormones and glucose, as measured from e.g., blood samples, urine samples and/or faecal samples.

### BRIEF DESCRIPTION OF THE FIGURES

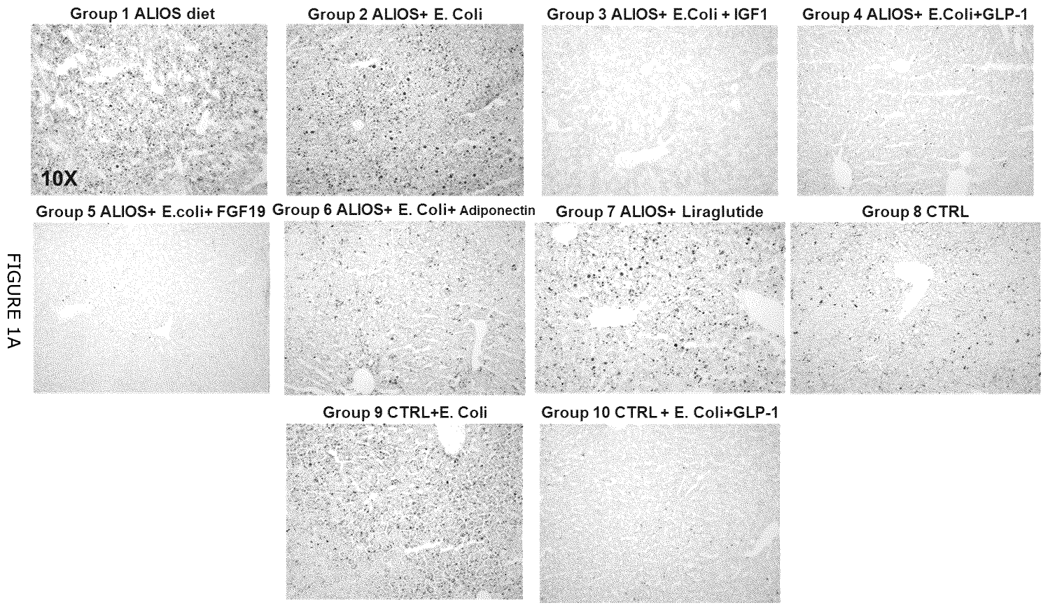

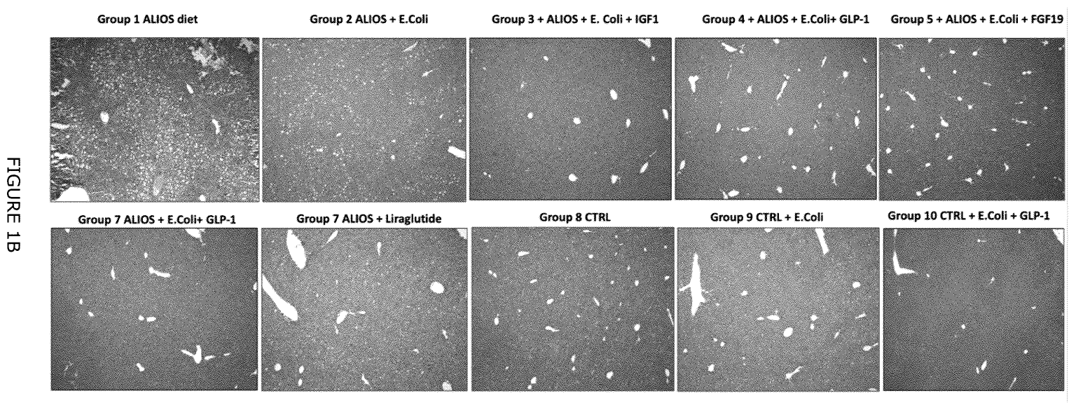

FIG. 1
A) ORO staining of left lateral lobe of mouse liver after 7 weeks of lifestyle modification intervention. (Group 1-6, and 8-10: n=6 per group; Group 7: n=5) (Fat droplets appear as black dots). Histological figures with ORO staining of the left lateral lobe of mouse liver showed that ALIOS mice Group 3 (E. coli+IGF1), 4 (E. coli+GLP-1) and 5 (E. coli+FGF19) did not show fat droplets in the liver while ALIOS mice Group 1 (ALIOS control), 2 (E. coli control) and 7 (Liraglutide) and Control diet mice Group 8 (Control diet only) and 9 (E. coli+GLP-1), had few fat droplets in the liver. B) H&E staining of left lateral lobe of mouse liver after 7 weeks of lifestyle modification intervention. (Group 1-6, and 8-10: n=6 per group; Group 7: n=5). The overall histological structure of the liver is preserved in all liver samples after 7 weeks of interventions. ALIOS mice Group 3 (E. coli+IGF1), 4 (E. coli+GLP-1) and 5 (E. coli+FGF19) did not show fat droplets in the liver compared to ALIOS mice Group 1 (ALIOS control) and 2 (E. coli control which small white droplets were detected.

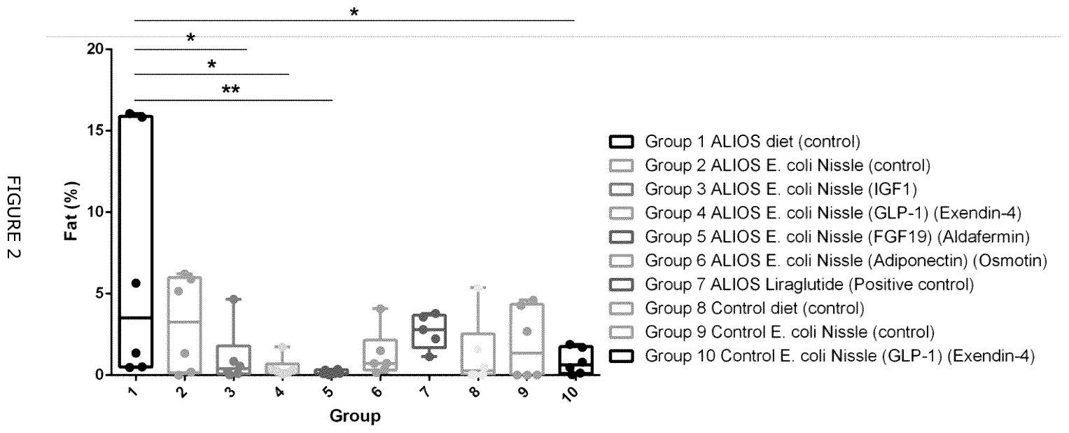

FIG. 2
Quantification of the liver fat droplets percentage from the ORO staining after 7 weeks of lifestyle modification intervention. *p≤0.05, **p≤0.01. (Group 1-6, and 8-10: n=6 per group; Group 7: n=5) Analysis done with Graphpad, Oneway-ANOVA, Tukey's multiple comparisons test. Quantification of the liver fat droplets percentage from the ORO staining showed that ALIOS mice Group 3 (E. coli+IGF1), 4 (E. coli+GLP-1), 5 (E. coli+FGF19) have significantly lower percentage of liver fat droplets compared with Group 1 (ALIOS control).

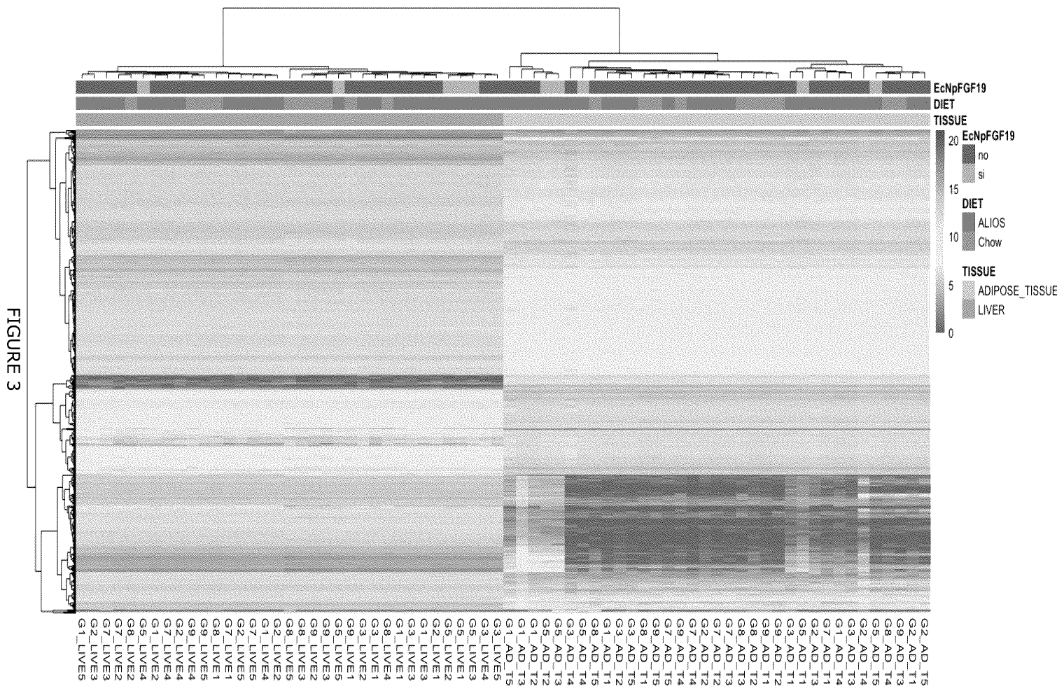

FIG. 3
Heat map showing first 1000 genes (Log2) differentially expressed based on the highest Standard deviation (SD) between the tissues (N=5 replicates per group). After 7 weeks of lifestyle interventions liver and epididymal adipose tissue showed clear different gene expression profiles. Three of five replicates belonging to Group 5 ALIOS+E. Coli FGF19 (EcNpFGF19 in figure) showed a clear cluster while two of them were separated in both liver and epididymal adipose tissue.

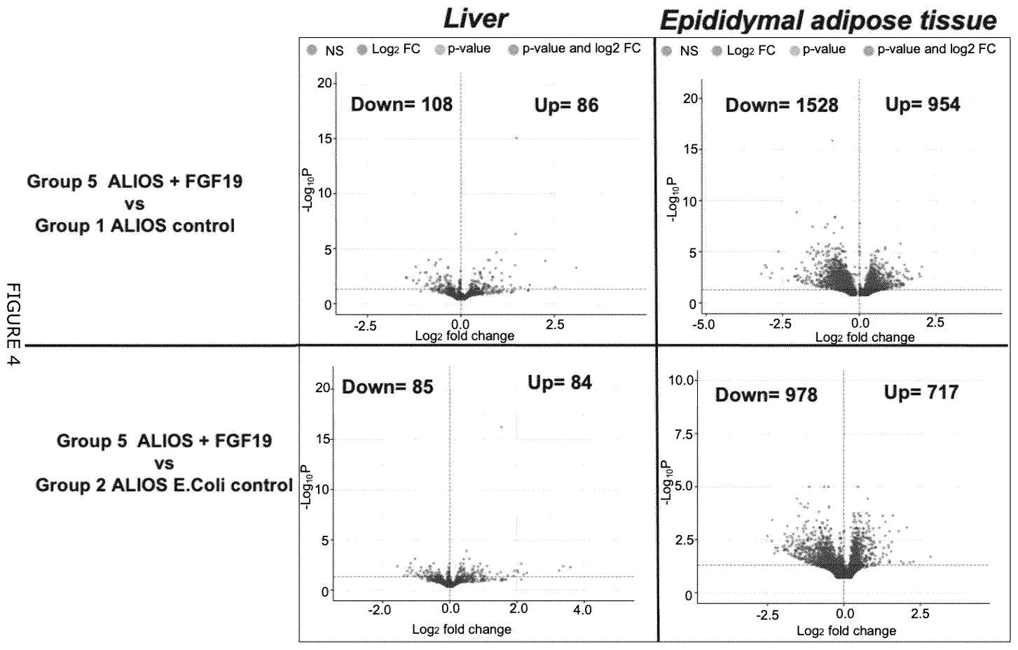

FIG. 4
Volcano plot showing abundance and number of differentially expressed genes (up and down expressed genes) in Group 5 ALIOS+FGF19 compared to Group 1 ALIOS control and Group 2 ALIOS E. Coli control. (FDR<0.05, FC10|). Analysis done using DESeq2.

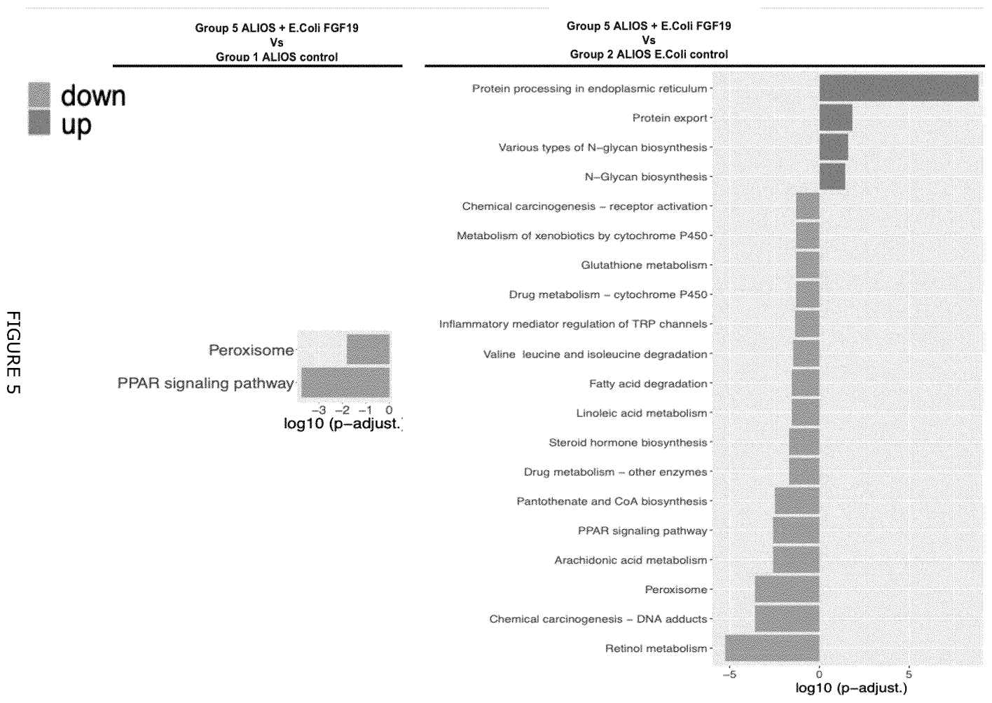

FIG. 5
Bar plot graphs showing up downregulated pathways in liver samples in Group 5 ALIOS+FGF19 compared to Group 1 ALIOS control and Group 2 ALIOS E.Coli control. Analysis done with Clusterprofiler (KEEG enrichment analysis) input for analysis: DGE padj (FDR)<0.05 and, FC |0|. Output: padj (FDR)<0.05 ((pvalueCutoff=0.05, pAdjustMethod=“BH).

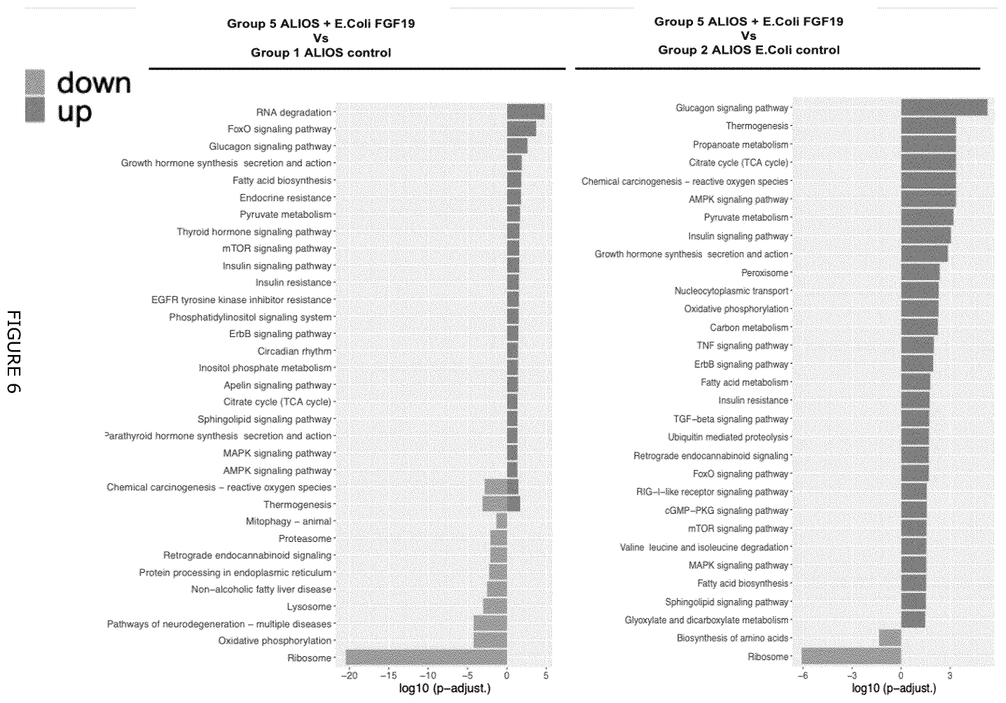

FIG. 6
Bar plot graphs showing up downregulated pathways in epididymal adipose tissue samples in Group 5 ALIOS+FGF19 compared to Group 1 ALIOS control and Group 2 ALIOS E.Coli control. Analysis done with Clusterprofiler (KEEG enrichment analysis) input for analysis: DGE padj (FDR)<0.05 and, FC |0|. Output: padj (FDR)<0.05 ((pvalueCutoff=0.05, pAdjustMethod=“BH).

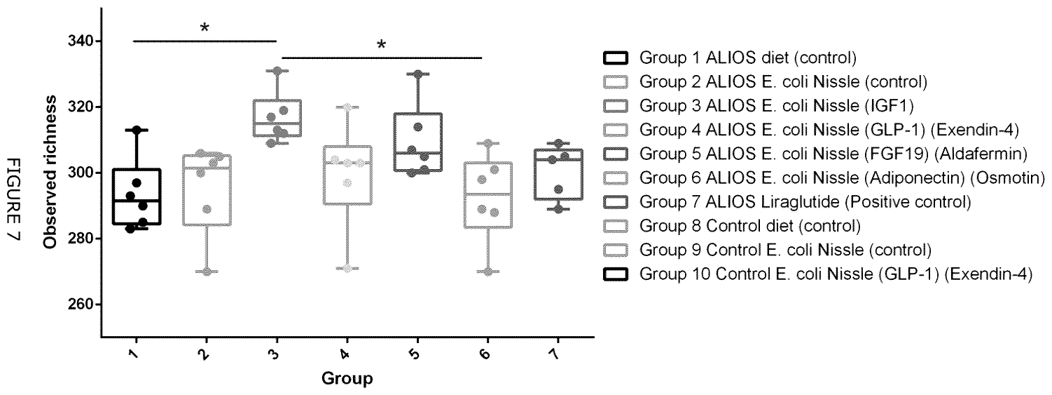

FIG. 7
Observed bacterial richness (16s sequencing). *p≤0.05. (Group 1-6, and 8-10: n=6 per group; Group 7: n=5) Analysis done with Graphpad, Oneway-ANOVA, Tukey's multiple comparisons test. ANOVA analysis of the observed richness showed that the different groups have significantly different alpha diversity (p-value: 0.011123; F-value: 3.3183) where ALIOS mice Group 3 (E. coli+IGF1) have significantly higher microbial richness compared with Group 1 (ALIOS control).

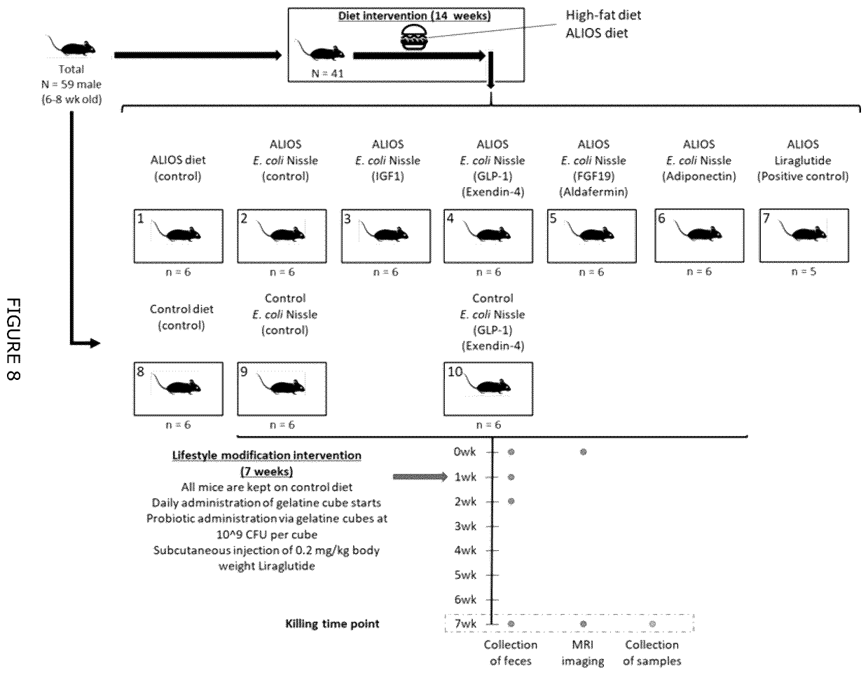

FIG. 8
Diagram for the intervention study. 60 mice were separated into 10 groups with in total 6 mice/group. During the diet intervention, 18 mice were fed the control diet and 42 mice fed by ALIOS diet for development of NAFLD for 14 weeks. The mice then underwent 7 weeks of lifestyle modification intervention while maintaining on control diet. ALIOS mice in Group 1 and Control mice in Group 8 were control groups where the mice were not given the probiotic treatment. ALIOS mice in Group 2 and Control mice in Group 9 were control groups for the hormonal intervention where the probiotics given did not produce any hormone. ALIOS mice in Groups 3 to 6 underwent probiotic intervention where the administered engineered E. coli Nissle 1917 strains expressed 4 different hormones (Group 3: IGF1; Group 4: GLP-1; Group 5: FGF19; Group 6: Adiponectin) respectively. Group 7 was the positive control group where Liraglutide was administered at 0.2 mg/kg body weight via subcutaneous injection. Control mice in Group 10 underwent probiotic intervention where the administered engineered E. coli Nissle 1917 strains expressed GLP-1.

### DETAILED DESCRIPTION

The present invention relates to a composition comprising a genetically engineered non-pathogenic microorganism expressing one or more polypeptides, wherein said polypeptides comprise polypeptide hormones selected from the group consisting of FGF-19, Aldafermin, IGF-1 and functional homologues of FGF-19, Aldafermin and IGF-1, and wherein FGF-19 has an amino acid sequence identified by SEQ ID NO: 1, Aldafermin has an amino acid sequence identified by SEQ ID NO: 2 and IGF-1 has an amino acid sequence identified by SEQ ID NO: 3, and a functional homologue thereof has a sequence identity of at least 80%, such as 85%, such as 90%, such as 95% or such as at least 99% towards SEQ ID NO: 1, 2 or 3.

Notably the present invention relates to a composition comprising a genetically engineered non-pathogenic microorganism expressing one or more polypeptides, comprising polypeptide hormones selected from the group consisting of FGF-19, Aldafermin and IGF-1.

The invention also relates to use of the composition in the treatment of fat liver and associated diseases such as non-alcoholic fat liver disease by use of a composition comprising non-pathogenic microorganism which expresses at least one polypeptide which comprises a polypeptide hormone. The benefit of a microorganism which expresses a polypeptide hormone is that it overcomes the current barriers involved in delivery of sustained levels of polypeptide hormones directly to the GI tract, without having to chemically modify the polypeptide hormones, that would otherwise be degraded within minutes following administration, and often before reaching the GI tract. The benefit of delivery by expression from a microorganism is that it is possible to obtain a sustained and constant delivery of the polypeptide hormones, which is highly beneficial in the treatment of non-alcoholic fat liver disease (NAFLD).

### Polypeptides

Polypeptide Hormones Peptide hormones also referred to in the present disclosure as polypeptide hormones, are a group of peptides that influence the endocrine system of animals, including humans. In mammalians peptide hormones are synthesized in cells from amino acids according to mRNA transcripts, which are synthesized from DNA templates inside the cell nucleus. Preprohormones, peptide hormone precursors, are then processed in several stages, typically in the endoplasmic reticulum, including removal of the N-terminal signal sequence and in some cases glycosylation, resulting in prohormones. The prohormones are then packaged into membrane-bound secretory vesicles, which can be secreted from the cell by exocytosis in response to specific stimuli (e.g., an increase in Ca²⁺ and cAMP concentration in cytoplasm). Following secretion, the peptide hormones can bind to a receptor on the surface of a cell inducing a second messenger response in the cell, which triggers signal transduction leading to the cellular responses. In some cases, such cellular responses promote cell survival and cell proliferation or at a physiological level, amongst other, promote growth or regulate mood and food craving. Thus, maintaining the peptide hormone homeostasis, i.e., a balanced peptide hormone level, is essential for hosts to maintain the steady internal, physical, and chemical conditions, required to maintain the optimal condition of the host. An unbalanced or maladapted peptide hormone homeostasis often leads to diseases, such as in the case of maladapted levels of FGF-19 or IGF-1 that in a host may be a significant contributing factor in the development of metabolic disorders, such as obesity associated diseases, in particular NAFLD.

In a pharmacological setting, peptide hormones such as Insulin, IGF-1, FGF-19, GLP-1 etc., are in general administered by subcutaneous injection, or in a few cases by oral delivery. Endogenous peptide hormones are usually short lived in in the acidic and peptidase rich environment of the digestive system and the bloodstream, which makes dosing difficult to predict and hampers their uses as long-acting treatments. The short lifetime and poor absorption after ingestion, have in some cases been overcome by introduction of multiple chemical modifications of the peptide hormones, overall complicating production and regulatory approval.

In NAFLD, the growth factors like FGF-19 and IGF-1 have reduced expression, and the continuous reduction in expression have been coupled to disease progression of NAFLD and promotes the disease progression towards NASH and liver cirrhosis. Thus, an enhanced expression of the polypeptide hormones of the present invention may facilitate a restoration of the growth factor expression level, that may hinder the disease progression of NAFLD and reverse the liver fat build up which is the hallmark of NAFLD.

Common for peptide hormones expressed in the genetically engineered microorganism, is their ability to reduce fat liver content in a host organism.

### FGF-19

Fibroblast growth factor 19 is a polypeptide that in humans is encoded by the FGF19 gene. It functions as a hormone, regulating bile acid synthesis, with effects on glucose and lipid metabolism. Reduced synthesis, and blood levels, may be a factor in chronic bile acid diarrhoea and in certain metabolic disorders. Patients with metabolic syndrome, non-alcoholic fatty liver disease and insulin resistance have reduced levels of FGF-19. FGF-19 increases to normal values in obese patients who undergo Roux-en-Y gastric bypass and other types of bariatric surgery.

Accordingly, in an embodiment of the invention, the polypeptide hormone expressed by the genetically engineered non-pathogenic microorganism is FGF-19. The absolute liver fat content of the host may be decreased following oral administration of composition of the invention, comprising a genetically engineered non-pathogenic microorganism expressing FGF-19.

In the present invention, FGF-19 is defined as a polypeptide according to SEQ ID NO: 1, or a functional homologue thereof with a sequence identity of at least 80%, such as 85%, such as 90%, such as 95% or such as at least 99% towards SEQ ID NO: 1.

Accordingly, FGF-19 may be encoded by a nucleic acid sequence according to SEQ ID NO: 4, the reverse complement thereof, or a nucleic acid sequence with a sequence identity of at least 80%, such as 85%, such as 90%, such as 95% or such as at least 99% towards SEQ ID NO: 4.

### Aldafermin

Aldafermin is an engineered analog of fibroblast growth factor 19 (FGF-19) that inhibits bile acid synthesis and regulates metabolic homeostasis, mimicking the effect of the endogenous analogue FGF-19, described above.

Thus, in an embodiment of the present invention, the polypeptide hormone expressed by the genetically engineered non-pathogenic microorganism is Aldafermin.

The absolute liver fat content of the host may be decreased following oral administration of the composition of the present invention, specifically comprising a genetically engineered non-pathogenic microorganism expressing Aldafermin.

In the present invention, Aldafermin is defined as a polypeptide according to SEQ ID NO: 2, or a functional homologue thereof with a sequence identity of at least 80%, such as 85%, such as 90%, such as 95% or such as at least 99% towards SEQ ID NO: 2.

Accordingly, Aldafermin may be encoded by a nucleic acid sequence according to SEQ ID NO: 5, the reverse complement thereof, or a nucleic acid sequence with a sequence identity of at least 80%, such as 85%, such as 90%, such as 95% or such as at least 99% towards SEQ ID NO: 5.

### IGF-1

Insulin-like growth factor 1 (IGF-1), also known as somatomedin C, is a peptide hormone similar in molecular structure to insulin and it plays an important role in childhood growth and has anabolic effects in adults. IGF-1 is a protein that in humans is encoded by the IGF1 gene. IGF-1 acts on, amongst others, the IGF-1 receptor, which in response to IGF-1 binding, induces the tyrosine autophosphorylation which triggers a cascade of intracellular signalling that, while cell type-specific, often promotes cell survival and cell proliferation. IGF-1 is used in clinics for treatment of growth failure in children, under the brand name of Increlex and iPlex, both administered by subcutaneous injection.

Thus, in an embodiment of the present invention, the polypeptide hormone expressed by the genetically engineered non-pathogenic microorganism is IGF-1.

The absolute liver fat content of the host may be decreased following oral administration of a composition of the present invention, specifically comprising a genetically engineered non-pathogenic microorganism expressing IGF-1.

In the present invention, IGF-1 is defined as a polypeptide according to SEQ ID NO: 3, or a functional homologue thereof with a sequence identity of at least 80%, such as 85%, such as 90%, such as 95% or such as at least 99% towards SEQ ID NO: 3.

Accordingly, IGF-1 may be encoded by a nucleic acid sequence according to SEQ ID NO: 6, the reverse complement thereof, or a nucleic acid sequence with a sequence identity of at least 80%, such as 85%, such as 90%, such as 95% or such as at least 99% towards SEQ ID NO: 6.

In embodiments of the invention a genetically engineered non-pathogenic microorganism expresses one or more polypeptides, wherein said polypeptides comprise polypeptide hormones selected from the group consisting of FGF-19 and functional homologues thereof, with an amino acid sequence identified by SEQ ID NO: 1, or a functional homologue thereof with a sequence identity of at least 80%, such as 85%, such as 90%, such as 95% or such as at least 99% towards SEQ ID NO: 1.

In embodiments of the invention a genetically engineered non-pathogenic microorganism expresses one or more polypeptides, wherein said polypeptides comprise polypeptide hormones selected from the group consisting of Aldafermin and functional homologues thereof, with an amino acid sequence identified by SEQ ID NO: 2, or a functional homologue thereof with a sequence identity of at least 80%, such as 85%, such as 90%, such as 95% or such as at least 99% towards SEQ ID NO: 2.

In embodiments of the invention a genetically engineered non-pathogenic microorganism expresses one or more polypeptides, wherein said polypeptides comprise polypeptide hormones selected from the group consisting of IGF-1 and functional homologues thereof, with an amino acid sequence identified by SEQ ID NO: 3, or a functional homologue thereof with a sequence identity of at least 80%, such as 85%, such as 90%, such as 95% or such as at least 99% towards SEQ ID NO: 3.

In further embodiments of the invention the one or more polypeptides are fusion peptide, chimeric peptide, and/or oligomeric peptide variants and/or combinations, of the polypeptide hormones FGF-19, Aldafermin and IGF-1.

Thus, in an embodiment of the invention the genetically engineered non-pathogenic microorganism expresses FGF-19, Aldafermin and/or IGF-1. In another embodiment the genetically engineered non-pathogenic microorganism of the present invention expresses FGF-19 and Aldafermin or functional homologues thereof. In another embodiment of the invention, the genetically engineered non-pathogenic microorganism expresses FGF-19 and IGF-1 or functional homologues thereof. In another embodiment the genetically engineered non-pathogenic microorganism of the present invention expresses Aldafermin and IFG-1 or functional homologues thereof.

In another embodiment of the invention the genetically engineered non-pathogenic microorganism expresses a polypeptide comprising FGF-19, Aldafermin and/or IGF-1 and at least one N-terminal polypeptide selected from the group consisting of polypeptides with an amino acid sequence of SEQ ID NOs: 7, 8, 9, 10, 11, 12, 13, 14, 15, 16 and 17, for promoting the excretion of said polypeptide from said genetically engineered non-pathogenic microorganism.

In embodiments the polypeptide expressed by the genetically engineered non-pathogenic microorganism is a fusion polypeptide comprises Aldafermin and IGF-1, comprising the amino acid sequence identified by SEQ ID NO: 2 and 3 or a functional homologue thereof with a sequence identity of at least 80%, such as 85%, such as 90%, such as 95% or such as at least 99% towards SEQ ID NO: 2 or 3.

In embodiments the polypeptide expressed by the genetically engineered non-pathogenic microorganism is a fusion polypeptide comprises two or more repetitions of the FGF-19, Aldafermin or IGF-1 polypeptide, identified by SEQ ID NO: 1, 2 or 3, respectively, or functional homologues thereof with a sequence identity of at least 80%, such as 85%, such as 90%, such as 95% or such as at least 99% towards SEQ ID NO: 1, 2 or 3.

In embodiments the polypeptide expressed by the genetically engineered non-pathogenic microorganism is a chimeric polypeptide comprises FGF-19, Aldafermin and/or IGF-1, with the amino acid sequence identified by SEQ ID NO: 1, 2 or 3, respectively, or a functional homologue thereof with a sequence identity of at least 80%, such as 85%, such as 90%, such as 95% or such as at least 99% towards SEQ ID NO: 1, 2 or 3.

### Polypeptide Variants

In the present disclosure, “polypeptides comprise/comprising polypeptide hormones” is to be understood as a polypeptide of a specific amino acid sequence, wherein the amino acid sequence of said polypeptide hormone(s) can be identified within the sequence. In the present invention “polypeptide hormones” may be a single polypeptide hormone or more than one polypeptide hormone, it may also refer to multiple repetitions of the same polypeptide hormone.

Variants of the polypeptide hormones of the invention, relates to sequence variants, i.e., variations in the amino acid sequence of the polypeptide hormones, as well as variants constructed by combining one or more polypeptide hormones, or by combination of one or more polypeptide hormone portions with one or more non-polypeptide hormone portions.

In that regard, the polypeptide in the context of the present invention may also be a fusion polypeptide recombining two or more polypeptides, wherein e.g., one part of a polypeptide is a signal peptide, and the second part of the polypeptide is a polypeptide hormone, such as FGF-19, Aldafermin or IGF-1. Alternatively, the polypeptide of the present invention may also be a fusion polypeptide recombining two or more polypeptide hormones i.e., an oligomeric polypeptide hormone. Thus, the fusion peptide may be a cis-fusion polypeptide, comprising two or more copies of the same polypeptide, or it may be a trans-fusion polypeptide/chimeric fusion polypeptide, comprising a first polypeptide hormone of the present invention, and a second polypeptide hormone of the present invention, wherein the first and second polypeptide hormone are not the same. Additionally, a trans-fusion polypeptide/chimeric polypeptide may comprise a polypeptide hormone portion and a non-polypeptide hormone portion. The non-polypeptide hormone portion may be a polypeptide that enhances the properties of the polypeptide hormone portion such as excretion, solubility, lifetime and cellular targeting properties. Regarding the non-polypeptide hormone portion, there are no limitations, except that for the present invention, the non-polypeptide hormone portion is a polypeptide comprising amino acids. Accordingly, non-limiting examples of non-polypeptide hormone portion are antibodies, and fragments thereof, such as but not limited to Fc-fragments, proteins, enzymes and peptides, with further options known to the person skilled in the art. Furthermore, the polypeptide hormone portion and the non-polypeptide hormone may be connected via an amino acid linker. Regarding the amino acid linker, there is no limitations and examples of suitable linker sequences are well known to the skilled person, non-limiting examples are described in Chen et al., Fusion Protein Linkers: Property, Design and Functionality, Adv Drug Deliv Rev. 2014.

When a polypeptide hormone is part of a polypeptide variant e.g, a fusion polypeptide, the non-polypeptide hormone part(s) is/are not part of the polypeptide hormone(s) as such and does not count when determining amino acid sequence identity as described herein. Consequently, when assessing the amino acid sequence identity of a peptide variant, such as a fusion polypeptide, the individual parts of the polypeptide, e.g., a polypeptide hormone part(s) and the non-polypeptide hormone part(s) are assessed individually. Thus, for a polypeptide comprising three parts, namely a signal peptide, and two polypeptide hormones, the sequence identity of each part should be addressed individually for each part. E.g., for a polypeptide comprising three parts, e.g., a signal peptide, FGF-19 and IGF-1, the sequence identity of each of the parts should evaluated individually, thus the sequence identify should be evaluated for the signal peptide, FGF-19 and IGF-1 individually.

Accordingly, the one or more polypeptides are fusion peptide, chimeric peptide, and/or oligomeric peptide variants and/or combinations, of FGF-19, Aldafermin and IGF-1.

In embodiments, the genetically engineered non-pathogenic microorganism expressing one or more polypeptides comprises at least 2, such as 3 polypeptide hormones selected from the group consisting of FGF-19, Aldafermin, IGF-1 and functional homologues of FGF-19, Aldafermin and IGF-1, and wherein FGF-19 has an amino acid sequence identified by SEQ ID NO: 1, Aldafermin has an amino acid sequence identified by SEQ ID NO: 2 and IGF-1 has an amino acid sequence identified by SEQ ID NO: 3, and a functional homologue thereof has a sequence identity of at least 80%, such as 85%, such as 90%, such as 95% or such as at least 99% towards SEQ ID NO: 1, 2 or 3.

Furthermore, in embodiments the genetically engineered non-pathogenic microorganism expressing one or more polypeptides comprises at least 2, such as 3, such as 4 repeated amino acid sequences of polypeptide hormones selected from the group consisting of FGF-19, Aldafermin, IGF-1 and functional homologues of FGF-19, Aldafermin and IGF-1, and wherein FGF-19 has an amino acid sequence identified by SEQ ID NO: 1, Aldafermin has an amino acid sequence identified by SEQ ID NO: 2 and IGF-1 has an amino acid sequence identified by SEQ ID NO: 3, and a functional homologue thereof has a sequence identity of at least 80%, such as 85%, such as 90%, such as 95% or such as at least 99% towards SEQ ID NO: 1, 2 or 3.

Additionally, in embodiments the genetically engineered non-pathogenic microorganism expressing one or more polypeptides, wherein said polypeptides comprise polypeptide hormones selected from the group consisting of FGF-19, Aldafermin, IGF-1 and functional homologues of FGF-19, Aldafermin and IGF-1, and wherein FGF-19 has an amino acid sequence identified by SEQ ID NO: 1, Aldafermin has an amino acid sequence identified by SEQ ID NO: 2 and IGF-1 has an amino acid sequence identified by SEQ ID NO: 3, and a functional homologue thereof has a sequence identity of at least 80%, such as 85%, such as 90%, such as 95% or such as at least 99% towards SEQ ID NO: 1, 2 or 3, further comprises one or more non-polypeptide hormone portions.

### A Signal Peptide

A polypeptide in the context of the present invention can e.g., comprise one or more polypeptide hormones selected from the group consisting of FGF-19, Aldafermin and IGF-1, and one or more signal peptides (a fusion polypeptide), wherein the signal peptide(s) can function to prompt the host cell to translocate the enzyme after expression in the cytosol. In general, a signal peptide may direct an expressed polypeptide to a specific cellular compartment and/or organelle, to the periplasm or signal for cellular excretion. In the present context, a signal peptide preferably directs the expressed polypeptide to cellular excretion. In E coli, there are multiple pathways that may be utilized for this purpose, such as the twin arginine translocation (TAT) pathway or the general secretory pathway (sec) pathway (see Tullman-Ecrcek et al., Export Pathway Selectivity of Escherichia coli Twin Arginine Translocation Signal Peptides, J B C, 2007).

A signal peptide is typically 16-100 amino acids long and present at the N-terminus of a newly synthesized polypeptide. In relation to the nucleic acid construct of the invention, the signal peptide is encoded by a nucleic acid sequence that may be operably linked to the heterologous nucleic acid sequence encoding a polypeptide of the invention. The signal peptide may also be a polypeptide tag, with an amino acid sequence comprising more than 30 amino acids.

The signal peptide acts as a polypeptide tag to promote selectively secretion of the polypeptide of the invention, e.g., a polypeptide tag that promotes the secretion of the polypeptide of the invention, i.e., promoting the secretion of FGF-19, Aldafermin, IGF-1, and homologues/variants thereof.

Accordingly, in embodiments, the polypeptide of the present invention further comprises an N-terminal polypeptide, such as a signal peptide, selected from the group consisting of phoA, lamb, ompA, fhuD, mdoD, ycdO, dsbA, usp45, egIA, nprM3, Alpha mating factor secretion signal, and functional homologues thereof.

Accordingly, in embodiments, the polypeptide of the present invention further comprises an N-terminal polypeptide, such as a signal peptide, selected from the group consisting of signal peptides with an amino acid sequence of SEQ ID NOs: 7, 8, 9, 10, 11, 12, 13, 14, 15, 16, 17 and functional homologues thereof.

The signal peptide is typically selected from the group consisting of, but not limited to, signal sequences listed in table 1 below.

TABLE 1
signal peptide sequences

| | | | SEQ |
|---|---|---|---|
| Signal sequence | Origin | Amino Acid Sequence | ID NO: |
| phoA | E. coli | MKQSTIALALLPLLFTPVTKA | 7 |
| lam B | E. coli | MMITLRKLPLAVAVAAGVMSAQAMA | 8 |
| ompA | E. coli | MKKTAIAIAVALAGFATVAQA | 9 |
| fhuD | E. coli | MSGLPLISRRRLLTAMALSPLLWQMNTAHA | 10 |
| mdoD | E. coli | MDRRRFIKGSMAMAAVCGTSGIASLFSQAAFA | 11 |
| ycdO | E. coli | MTINFRRNALQLSVAALFSSAFMANA | 12 |
| dsbA | E. coli | MKKIWLALAGLVLAFSASA | 13 |
| usp45 | L. lactis | MKKKIISAILMSTVILSAAAPLSGVYA | 14 |
| eglA | C. saccharobutylicum | MFSKIKKINFFKKTFSFLIAVVMMLFTVLGTNTYKAEA | 15 |
| nprM3 | C. sporogenes | MKSKKLLATVLSAVITLSTVSAVYA | 16 |
| Alpha mating factor | S. boulardii | MRFPSIFTAVLFAASSALAAPVNTTTEDETAQIPA | 17 |
| secretion signal | | EAVIGYSDLEGDFDVAVLPFSNSTNNGLLFINTTI | |
| | | ASIAAKEEGVSLDKREEGEPK | |

In an embodiment wherein the heterologous polypeptide is part of a polypeptide comprising a signal sequence, the signal sequence is not part of the heterologous polypeptide as such and does thus not count when determining amino acid sequence identity as described herein.

### Functional Homologue

A functional homologue or functional variant of a protein/nucleic acid sequence as described herein is a protein/nucleic acid sequence with alterations in the genetic code, which retain its original functionality. A functional homologue may be obtained by mutagenesis or may be natural occurring variants from the same or other species. The functional homologue should have a remaining functionality of at least 50%, such as 60%, 70%, 80%, 90% or 100% compared to the functionality of the protein/nucleic acid sequence.

A functional homologue of any one of the disclosed amino acid or nucleic acid sequences can also have a higher functionality. A functional homologue of any one of the amino acid sequences shown in any of tables 1, 2 or 3, or a nucleic acid sequence encoding these sequences or amino acid or nucleic acid sequences as disclosed in SEQ ID NOs: 1-23, should ideally be able to participate in the alleviation and/or treatment of NAFLD in the host, in terms of e.g., increased efficacy, enhanced export of the polypeptide out of the cell or enhanced viability of the genetically engineered non-pathogenic microorganism.

### Sequence Identity

The term “sequence identity” as used herein describes the relatedness between two amino acid sequences or between two nucleotide sequences, i.e., a polypeptide hormone sequence (e.g., a sequence of the invention) and a reference sequence (such as a prior art sequence) based on their pairwise alignment. For purposes of the present invention, the sequence identity between two amino acid sequences is determined using the Needleman-Wunsch algorithm (Needleman and Wunsch, 1970, J. Mo/. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later (available at https://www.ebi.ac.uk/Tools/psa/emboss needle/). The parameters used are gap open penalty of 10, gap extension penalty of 0.5, and the EBLOSUM62 (EMBOSS version of 30 BLOSUM62) substitution matrix. The output of Needle labelled “longest identity” (obtained using the -nobrief option) is used as the percent identity and is calculated as follows: (Identical Residuesχ 100)/(Length of Alignment−Total Number of Gaps in Alignment)

For purposes of the present invention, the sequence identity between two nucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, Trends Genet. 16: 276-277), 10 preferably version 5.0.0 or later. The parameters used are gap open penalty of 10, gap extension penalty of 0.5, and the DNAFULL (EMBOSS version of NCBI NUC4.4) substitution matrix. The output of Needle labelled “longest identity” (obtained using the -no brief option) is used as the percent identity and is calculated as follows: (Identical Deoxyribonucleotides×100)/(Length of Alignment−Total Number of Gaps in Alignment).

### A Genetically Engineered Non-Pathogenic Microorganism

In the present context, the terms “a genetically modified non-pathogenic microorganism” and “a genetically engineered non-pathogenic microorganism” are used interchangeably. As used herein “a genetically modified non-pathogenic microorganism” is a host cell whose genetic material has been altered by human intervention using a genetic engineering technique, such a technique is for example but not limited to transformation or transfection e.g., with a heterologous polynucleotide sequence, Crisper/Cas editing and/or random mutagenesis. In one embodiment the genetically engineered cell has been transformed or transfected with a recombinant nucleic acid sequence.

Genetically modified non-pathogenic microorganisms can in general contain one or more genes that are not present in the native (not genetically engineered) form of the microorganism. Techniques for introducing exogenous nucleic acid molecules/sequences and/or inserting exogenous nucleic acid molecules/sequences (recombinant, heterologous) into a cell's hereditary information for inserting, deleting or altering the nucleic acid sequence of a cell's genetic information are known to the skilled artisan.

A genetically non-pathogenic microorganism can contain one or more nucleic acid sequences and/or genes that are present in the native form of the cell, wherein said nucleic acid sequences and/or genes are modified and re-introduced into the microbial cell by artificial means.

The term “genetically engineered” also encompasses cells that contain a nucleic acid molecule being endogenous to the cell that has been modified without removing the nucleic acid molecule from the cell.

Such modifications include those obtained by gene replacement, site-specific mutations, and related techniques.

Genetically engineered microorganisms of the invention can be provided using standard methods of the art e.g., those described in the manuals by Sambrook et al., Wilson and Walker, Maniatis et al., and Ausubel et al.

### Non-Pathogenic Microorganism

Regarding the microorganism, there are, in principle, no limitations; they may be eubacteria (gram-positive or gram-negative) or archaebacteria, as long as they allow genetic manipulation for insertion of a gene of interest and can be cultivated on a manufacturing scale, while being non-pathogenic to the host. The genetically engineered non-pathogenic microorganism is preferably a prokaryotic cell, such as a microbial cell. Appropriate microbial cells that may function as a host cell include yeast cells, bacterial cells, archaebacterial cells, algae cells, and fungal cells.

In embodiments, the non-pathogenic microorganism is a yeast or bacteria.

The genetically modified non-pathogenic microorganism may be e.g., a bacterial or yeast cell. In one preferred embodiment, the genetically modified non-pathogenic microorganism is a bacterial cell. The non-pathogenic microorganism may be any microorganisms that, when administered in adequate amounts, confer a health benefit on the host, examples of such are members of the Lactobacillus species, members of the Bifidobacterium species, Streptococcus thermophilus, Escherichia coli (E. coli), Bacillus cereus, Clostridum butyricum, Enterococcus faecalis, Enterococcus faecium, Saccharomyces boulardii, Saccharaomyces cerevisiae and strains derived thereof.

In embodiments of the present invention, the non-pathogenic microorganism is a microorganism of a genus selected from of the group consisting of Escherichia, Bacteroides, Clostridiales, Bifidobacteriales, Eubacteriales and Lactobacillales.

In further embodiments of the present invention, the non-pathogenic microorganism is a species selected from of the genus group consisting of Escherichia coli, Anaerobutyricum soehngenii, Lactococcus lactis, Bacteroides thetaiotaomicron, Bacteroides oavtus, Bacteroides xylanisolvens and Clostridium symbiosum.

Several non-pathogenic strains are used in production of dairy products and dietary supplements, such as Lactobacillus bulgaricus and Streptococcus thermophilus, but also non-pathogenic strains of E. coli have gained impasse in the production of probiotic dietary supplements and in prophylactic treatment and in treatment of disease, the most well studied example of a non-pathogenic E. coli strain is E. coli. Nissle 1917 Thus, in an embodiment of the invention the non-pathogenic microorganism is E. coli. In further embodiments of the present invention the non-pathogenic microorganism is selected from the group consisting of Escherichia coli Nissle 1917, Escherichia coli Symbioflor G1/2, Escherichia coli Symbioflor G4/9, Escherichia coli Symbioflor G5, Escherichia coli Symbioflor G6/7 and Escherichia coli Symbioflor G8. In an embodiment of the invention the non-pathogenic microorganism is Escherichia coli.

In a preferred embodiment of the invention the non-pathogenic microorganism is E. coli Nissle 1917. In embodiments, the non-pathogenic microorganism is selected from member of the Anaerobutyricum genus, preferably, Anaerobutyricum soehngenii L2-7. In other embodiments, the non-pathogenic microorganism is selected from members of the Lactococcus genus, preferably, Lactococcus lactis mg1363.

In another embodiment of the invention the non-pathogenic microorganism is Saccharaomyces cerevisiae. In a preferred embodiment of the present invention the non-pathogenic microorganism is Saccharomyces boulardii.

### A Composition Comprising Said Cell or Combination of Cells

The present invention particularly relates to a composition comprising a genetically engineered non-pathogenic microorganism expressing one or more polypeptides, wherein said polypeptides comprise polypeptide hormones.

Thus, in embodiments, the invention relates to a composition comprising a genetically engineered non-pathogenic expressing one or more polypeptides, wherein said polypeptides comprise polypeptide hormones selected from the group consisting of FGF-19, Aldafermin, IGF-1 and functional homologues thereof as described herein.

In an embodiment, the present invention relates to a composition comprising a genetically engineered non-pathogenic microorganism expressing one or more polypeptides, wherein said polypeptides comprise polypeptide hormones selected from the group consisting of FGF-19, Aldafermin, IGF-1 and functional homologues of FGF-19, Aldafermin and IGF-1, and wherein FGF-19 has an amino acid sequence identified by SEQ ID NO: 1, Aldafermin has an amino acid sequence identified by SEQ ID NO: 2 and IGF-1 has an amino acid sequence identified by SEQ ID NO: 3, and a functional homologue thereof has a sequence identity of at least 80%, such as 85%, such as 90%, such as 95% or such as at least 99% towards SEQ ID NO: 1, 2 or 3.

The composition of the invention may be a therapeutic composition.

### Combinations of Microorganisms

In embodiments, the composition of the present invention comprises more than one genetically engineered non-pathogenic microorganism expressing one or more polypeptides, wherein said polypeptides comprise polypeptide hormones selected from the group consisting of FGF-19, Aldafermin, IGF-1 and functional homologues thereof as described herein. As a non-limiting example, three microorganisms are all part of a composition of the present invention, wherein one genetically engineered non-pathogenic microorganism may express FGF-19 or functional homologues thereof, while another genetically engineered non-pathogenic microorganism of the present invention may express IGF-1 or functional homologues thereof and a third genetically engineered non-pathogenic microorganism may express Aldafermin. Alternatively, the composition of the present invention may comprise at least one genetically engineered non-pathogenic microorganism, wherein one or more expressing one or more polypeptides, wherein said polypeptides comprise polypeptide hormones selected from the group consisting of FGF-19, Aldafermin, IGF-1 and homologues thereof, and at least one other non-pathogenic microorganism. Regarding the at least one other non-pathogenic microorganism there is no limitations, and the at least one other non-pathogenic microorganism may e.g., be a probiotic organism such as but not limited to Lactobacillus, Bifidobacterium, Lactococcus lactis, Anaerobutyricum soehngenii, Saccharomyces boulardii, Lactic acid bacteria such as but not limited to Lactobacillus planatrum, Lacobacillys fermentum, Lactobacillus helveticus, Lactobacillus, Lactobacillus rhamnosus and Lactobacillus acidophilus, and bacterial species such as but not limited to Bacteroides thetaiotaomicron, Bacteroides xylanisolvens, Bacteroides ovatus and Bacteroides dorei.

### A Recombinant Nucleic Acid Construct

The present invention also provides a nucleic acid construct comprising a recombinant nucleic acid sequence encoding one or more polypeptides of the invention, wherein the recombinant nucleic acid sequence may be flanked by regulatory elements that regulate the expression of said polypeptides. Accordingly, a nucleic acid construct of the present invention comprises a nucleic acid sequence encoding a polypeptide of the present invention with a nucleic acid sequence according to SEQ ID NO: 4, 5 or 6, or a functional homologue thereof with a sequence identity of at least 70%, such as 80%, such as 90%, such as 95% or such as at least 99% towards SEQ ID NO: 4, 5 or 6, or the reverse complement thereof. In addition, said nucleic acid construct may also comprise a non-coding nucleic acid sequence regulating the expression of said polypeptide.

A nucleic acid sequence comprising both the coding and non-coding sequences may also be referred to as a recombinant nucleic acid sequence.

In the present context, the term “recombinant nucleic acid sequence” or “recombinant gene/nucleic acid/nucleotide sequence/DNA encoding” are used interchangeably and intended to mean an artificial nucleic acid sequence (i.e. produced in vitro using standard laboratory methods for making nucleic acid sequences) that comprises a set of consecutive, non-overlapping triplets (codons) which is transcribed into mRNA and translated into a protein when under the control of the appropriate control sequences, i.e. a promoter sequence.

The boundaries of the coding sequence are generally determined by a ribosome binding site located just upstream of the open reading frame at the 5′end of the mRNA, a transcriptional start codon (AUG, GUG or UUG), and a translational stop codon (UAA, UGA or UAG). A coding sequence can include, but is not limited to, genomic DNA, cDNA, synthetic, and recombinant nucleic acid sequences.

The term “nucleic acid” includes RNA, DNA and cDNA molecules. It is understood that, as a result of the degeneracy of the genetic code, a multitude of nucleic acid sequences encoding a given protein may be produced.

The recombinant nucleic acid sequence may be a coding DNA sequence e.g., a gene, or non-coding DNA sequence e.g., a regulatory DNA, such as a promoter sequence.

In embodiments of the invention, the nucleic acid sequence encoding the one or more polypeptides is optimized for expression in the specific non-pathogenic microorganism.

The recombinant nucleic acid sequence may also be heterologous. As used herein “heterologous” refers to a polypeptide, amino acid sequence, nucleic acid sequence or nucleotide sequence that is foreign to a cell or organism, i.e., to a polypeptide, amino acid sequence, nucleic acid molecule or nucleotide sequence that does not naturally occurs in said cell or organism.

Thus, a nucleic acid construct provided by the present invention comprises a coding nucleic sequence, i.e. recombinant DNA sequence of a gene of interest, e.g., an IFG-1 gene, and a non-coding regulatory nucleic acid sequence, e.g., a promoter DNA sequence, e.g., a recombinant promoter sequence derived from the Schanzetta promoter sequences (see Armetta et al., Escherichia coli promoters with consistent expression throughout the murine gut, ACS synthetic biology, 2021), or a promoter sequence derived from another genomic promoter sequence, or a synthetic promoter sequence. Preferably, the promoter elements are selected promoters with a sequence according to any one of SEQ ID NOs: 18, 19, 20, 21, 22 and 23. In embodiments, the promoters are selected from Schanzetta promoters of SEQ ID NOs: 18, 19 and 20, and preferably Schanzetta promoter 8 with a nucleic acid sequence according to SEQ ID NO: 18. Preferably, the polypeptide encoding sequence is under the control of a promoter sequence selected from promotor sequences with a nucleic acid sequence as identified in Table 2.

TABLE 2
Promoter sequences

| | | | SEQ |
|---|---|---|---|
| Promoter name | Sequence | Reference | ID NO: |
| Schanzetta | TGCTTGACTCGTCGTTATCCTACGTGTATAAT | Armetta et al. 2021 | 18 |
| Promoter 8 | TGGC | | |
| Schanzetta | TGCTTGACTCGTCGTTCCTCCTACGTGTATA | Armetta et al. 2021 | 19 |
| Promoter 7 | ATTGG | | |
| Schanzetta | TGCTGGACTCGTCGTAATCCTGCGTGTATAA | Armetta et al. 2021 | 20 |
| Promoter 6 | TTGGC | | |
| J23107 | TTTACGGCTAGCTCAGCCCTAGGTATTATGC | iGEM's Anderson promoter library, see | 21 |
| | TAGC | http://parts.igem.org/Promoters/ | |
| | | Catalog/Anderson | |
| TDH3 | CGAGTTTATCATTATCAATACTGCCATTTCAA | Partow et al. 2010, Characterization | 22 |
| (yeast promoter) | AGAATACGTAAATAATTAATAGTAGTGATTTT | of different promoters for designing a | |
| | CCTAACTTTATTTAGTCAAAAAATTAGCCTTT | new expression vector in | |
| | TAATTCTGCTGTAACCCGTACATGCCCAAAA | Saccharomyces cerevisiae, Yeast, | |
| | TAGGGGGGGGGTTACACAGAATATATAACAT | 27(11): 955-64 | |
| | CGTAGGTGTCTGGGTGAACAGTTTATTCCTG | | |
| | GCATCCACTAAATATAATGGAGCCCGCTTTT | | |
| | TAAGCTGGCATCCAGAAAAAAAAAGAATCCC | | |
| | AGCACCAAAATATTGTTTTCTTCACCAACCAT | | |
| | CAGTTCATAGGTCCATTCTCTTAGCGCAACT | | |
| | ACAGAGAACAGGGGCACAAACAGGCAAAAA | | |
| | ACGGGCACAACCTCAATGGAGTGATGCAAC | | |
| | CTGCCTGGAGTAAATGATGACACAAGGCAAT | | |
| | TGACCCACGCATGTATCTATCTCATTTTCTTA | | |
| | CACCTTCTATTACCTTCTGCTCTCTCTGATTT | | |
| | GGAAAAAGCTGAAAAAAAAGGTTGAAACCAG | | |
| | TTCCCTGAAATTATTCCCCTACTTGACTAATA | | |
| | AGTATATAAAGACGGTAGGTATTGATTGTAAT | | |
| | TCTGTAAATCTATTTCTTAAACTTCTTAAATTC | | |
| | TACTTTTATAGTTAGTCTTTTTTTTAGTTTTAA | | |
| | AACACCAAGAACTTAGTTTCGAATAAACACA | | |
| | CATAAACAAACAAA | | |
| TEF1 | CAAAATGTTTCTACTCCTTTTTTACTCTTCCA | Partow et al. 2010, Characterization | 23 |
| (yeast promoter) | GATTTTCTCGGACTCCGCGCATCGCCGTACC | of different promoters for designing a | |
| | ACTTCAAAACACCCAAGCACAGCATACTAAA | new expression vector in | |
| | TTTCCCCTCTTTCTTCCTCTAGGGTGTCGTTA | Saccharomyces cerevisiae, Yeast, | |
| | ATTACCCGTACTAAAGGTTTGGAAAAGAAAA | 27(11): 955-64 | |
| | AAGAGACCGCCTCGTTTCTTTTTCTTCGTCG | | |
| | AAAAAGGCAATAAAAATTTTTATCACGTTTCT | | |
| | TTTTCTTGAAAATTTTTTTTTTTGATTTTTTTCT | | |
| | CTTTCGATGACCTCCCATTGATATTTAAGTTA | | |
| | ATAAACGGTCTTCAATTTCTCAAGTTTCAGTT | | |
| | TCATTTTTCTTGTTCTATTACAACTTTTTTTAC | | |
| | TTCTTGCTCATTAGAAAGAAAGCATAGCAAT | | |
| | CTAATCTAAGTTTTAATTACAAA | | |

The term “nucleic acid construct” means an artificially constructed segment of nucleic acids, in particular a DNA segment, which is intended to be inserted into a target cell, e.g., a bacterial cell, to modify expression of a gene of the genome or express a gene/coding DNA sequence which may be included in the construct.

Furthermore, the coding sequence and the promoter sequences are preferably operably linked.

The term “operably linked” refers to a functional relationship between two or more nucleic acid (e.g., DNA) segments. operably linked refers to the functional relationship of a transcriptional regulatory sequence to a transcribed sequence. For example, a promoter sequence is operably linked to a coding sequence if it stimulates or modulates the transcription of the coding sequence in an appropriate host cell or other expression system. In one embodiment of the invention the cluster of recombinant nucleic acid sequences encoding a transporter protein are operably linked such that they are transcribed by a single promoter sequence.

Generally, promoter sequences that are operably linked to a transcribed sequence are physically contiguous to the transcribed sequence, i.e., they are cis-acting.

In one exemplified embodiment, the nucleic acid construct of the invention may be a part of vector DNA e.g., a plasmid, such as pMUT1, in another embodiment, the construct it is an expression cassette/cartridge that is integrated in the genome of a host cell.

Thus, in embodiments the present invention provides a recombinant nucleic acid construct comprising a nucleic acid sequence according to SEQ ID NO: 4, 5 and/or 6, or a functional homologue thereof with a sequence identity of at least 70%, such as 80%, such as 90%, such as 95% or such as at least 99% towards SEQ ID NO: 4, 5 or 6, or the reverse complement thereof, encoding a polypeptide of the invention.

In a further embodiment, the nucleic acid construct of the present invention further one or more promoter elements, modulating the expression of said one or more polypeptides, such as a promoter element selected from the group consisting of 18, 19, 20, 21, 22 and 23, preferably a Schazetta promoter element according to the nucleic acid sequence of SEQ ID NO: 18, or the reverse complement thereof.

In a further embodiment of the invention, the nucleic acid construct of the present invention is comprised in an expression vector.

In further embodiments an expression vector and/or construct is integrated into the genome of the genetically engineered non-pathogenic microorganism. Integration of the nucleic acid construct of interest comprised in the construct (expression cassette) into the bacterial genome can be achieved by conventional methods, e.g. by using linear cartridges that contain flanking sequences homologous to a specific site on the chromosome, as described for the attTn7-site (Waddell C. S. and Craig N. L., Genes Dev. (1988) Feb;2(2):137-49); methods for genomic integration of nucleic acid sequences in which recombination is mediated by the Red recombinase function of the phage A or the RecE/RecT recombinase function of the Rac prophage (Murphy, J Bacteriol. (1998);180(8):2063-7; Zhang et al., Nature Genetics (1998) 20: 123-128 Muyrers et al., EMBO Rep. (2000) 1(3): 239-243); methods based on Red/ET recombination (Wenzel et al., Chem Biol. (2005), 12(3):349-56; Vetcher et al., Appl Environ Microbiol. (2005);71(4):1829-35); or positive clones, i.e., clones that carry the expression cassette, can be selected e.g., by means of a marker gene, or loss or gain of gene function.

In another embodiment the nucleic acid construct is integrated into a plasmid. A plasmid of the present invention may be an endogenous, exogeneous or artificial plasmid, and preferably, the plasmid is an endogenous plasmid, such as the pMUT1 plasmid of E. coli.

The nucleic acid construct or plasmid may be integrated into the genetically engineered non-pathogenic microorganism by any method known to the skilled person, and preferably, integration is done by transformation.

Accordingly, the present invention relates to a composition comprising a genetically engineered non-pathogenic microorganism comprising a nucleic acid construct encoding a polypeptide of the invention.

### A Process for Preparing/Producing a Composition Comprising a Genetically Engineered Non-Pathogenic Microorganism

The invention also provides a process for preparing a composition comprising a genetically engineered non-pathogenic microorganism.

The process for preparing a genetically engineered non-pathogenic microorganism can comprise a step for preparing the non-pathogenic microorganism such as E. coli Nissle 1917 for transformation with a plasmid or for genetic integration. The process furthermore contains a step for preparing the plasmid, construct or gene for transformation or genomic integration. The process could include a step for transforming the non-pathogenic microorganism with a plasmid or construct, encoding one or more polypeptides of the invention. Alternatively, the process could contain a step for integrating the gene encoding the one or more polypeptides comprising polypeptide hormones, an optionally a signal peptide as well as one or more promoter sequences, into the genome of the non-pathogenic microorganism. The process also contains a step for selecting the genetically engineered non-pathogenic microorganism over a non-genetically engineered non-pathogenic microorganism. Said selection step could comprise, antibiotic selection or nutritional selection. The genetically engineered non-pathogenic microorganism can be subjected to validation in order to confirm their genus/species identity, the absence of pathogenic toxins and susceptibility to clinically used antibiotics. For example, using PCR, qPCR, next-generation sequencing and/or Sanger sequencing, e.g., of all or part of the genome sequence, can be performed to confirm genus/species identity.

To ensure that the genetically engineered non-pathogenic microorganism is excreted from the subject, or in the case of side effects, the non-pathogenic microorganism may be tested for antibiotic resistance prior to administration to a subject.

Thus, in embodiments of the invention the genetically engineered non-pathogenic microorganism is confirmed to be sensitive or susceptible (e.g., lack resistance) to one or more antibiotic agents selected from the group consisting of antibiotic macrolides (e.g., azithromycin, clarithromycin, erythromycin, fidaxomicin, telithromycin, carbomycin A, josamycin, kitasamycin, midecamycin/midecamycin acetate, oleandomycin, solithromycin, spiramycin, troleandomycin, tylosin/tylocine, or roxithromycin), rifamycins (e.g., rifampicin (or rifampin), rifabutin, rifapentine, rifalazil, or rifaximin), polymyxins (e.g., polymyxin B, or polymyxin E (colistin)), quinolone antibiotics (e.g., nalidixic acid, ofloxacin, levofloxacin, ciprofloxacin, norfloxacin, enoxacin, lomefloxacin, grepafloxacin, trovafloxacin, sparfloxacin, temafloxacin, moxifloxacin, gatifloxacin, or gemifloxacin), β-lactams (e.g., penicillin, cloxacillin, dicloxacillin, flucloxacillin, methicillin, nafcillin, oxacillin, temocillin, amoxicillin, ampicillin, mecillinam, carbenicillin, ticarcillin, azlocillin, mezlocillin, or piperacillin), aminoglycosides (e.g., amikacin, gentamicin, neomycin, streptomycin, or tobramycin), cephalosporins (e.g., cefadroxil, cefazolin, cephalexin, cefaclor, cefoxitin, cefprozil, cefuroxime, loracarbef, cefixime, cefdinir, cefditoren, cefoperazone, cefotaxime, cefpodoxime, cefepime, or ceftobiprole), monobactams (e.g., aztreonam, tigemonam, nocardicin A, or tabtoxinine β-lactam), carbapenems (e.g., biapenem, doripenem, ertapenem, faropenem, imipenem, meropenem, panipenem, razupenem, tebipenem, or thienamycin), and/or tetracyclines (e.g., tetracycline, chlortetracycline, oxytetracycline, demeclocycline, lymecycline, meclocycline, methacycline, minocycline, rolitetracycline, or tigecycline). Generally, transformation of a purified native microorganism with a heterologous polynucleotide confers antibiotic resistance to one or more antibiotic agents used for selection of transformed non-pathogenic microorganisms, e.g., resistance to kanamycin, chloramphenicol, carbenicillin, hygromycin and/or trimethoprim. Genetic integration of the heterologous polynucleotide into the genome of the non-pathogenic microorganism, can in one aspect of the invention confer antibiotic resistance to one or more antibiotic agents used for selection of the genetically engineered non-pathogenic microorganism. In another aspect the invention the genetic integration, of the heterologous polynucleotide into the genome of the non-pathogenic microorganism, does not confer antibiotic resistance to one or more antibiotic agents used for selection of the genetically engineered non-pathogenic microorganism.

The process also includes a step for culturing the genetically engineered non-pathogenic microorganism, such as in a shaker culture or in a bioreactor, such as a fermenter or bacterial culture tank, or in a sponge culture. Following cultivation, the process contains a step for harvesting the genetically engineered non-pathogenic microorganism, the harvesting method could be any common method for harvesting biological cultures, such as centrifugation, microfiltration, membrane cross flow microfiltration, ultrafiltration, harvest by viafuge, sedimentation or flocculation, freeze drying and/or direct spray drying.

The process might also contain a combination of the above methods for harvesting. The harvested genetically engineered non-pathogenic microorganism may be stored, in any common manner known to the skilled person, and/or further processed in order to prepare the cell for administration. Further processing could contain drying, granulation, powdering, micronization, resuspension or other methods known to the skilled person.

The composition comprising a genetically engineered non-pathogenic microorganism of the present invention may be delivered as a lyophilized (freeze-dried) powder packaged in a consumable capsule. One process for preparing a lyophilized powder of the composition of the invention may be prepared as described in brief; the liquid culture can be: centrifuged, resuspended in a lyophilization medium which optionally can include cryoprotectants and biological- and/or chemical-oxygen scavengers transferred under anaerobic conditions to a lyophilizer, lyophilized, encapsulated in a capsule under anaerobic conditions, and packaged in a glass ampoule to maintain oxygen free conditions during transport and storage. The robustness of the composition over time can be assessed using different configurations containing single and various factorial mixtures of excipients prepared via the same lyophilization, encapsulation, and packaging procedures. Products can then be stored in a laboratory setting on a shelf at room temperature, in a refrigerator or in a freezer and tested for viability at 0, 30, 60, 180, and 360 days from the date of production. Validation can be performed by breaking an ampoule under aerobic conditions (as would be encountered when delivering the capsule to a subject in a medical setting) and then placing the capsule in a suitable media.

### Use in Modulation of Gut Microbiome

The gut microbiome is a complex composition of many different microorganisms that are entangled in the sense that they regulate each other through competition for the same nutrients and occupational space. The present invention in that regard relates to a use of the composition according to the present invention in the modulation of the gut microbiome of the host. Example 5 showed how a composition of the present invention may modulate the gut microbiome of a host organism following repeated administration of said composition to said host. In that regard a composition of the present invention may be administered once or repeatedly to the host. Thus, in an embodiment the composition of the present invention is administered once and, in another embodiment, said composition is administered repeatedly.

A microbiome is the community of microorganisms that can usually be found living together in any given habitat. In the present disclosure, microbiome refers to the community of microorganisms which is found in the gut of a host organism.

In general, the diversity of a host's gut microbiome is described by a single identifier, namely the alpha diversity which is a measure of microbiome diversity applicable to a single sample. In general, a high alpha diversity is seen as beneficial for the host and many known medications such as antibiotics influences the alfa diversity in a negative way, by reducing the alpha diversity due to the antibiotic effect. Nevertheless, the alpha diversity is a guidance value, as upregulation of one beneficial microorganism might be beneficial, while upregulation of a pathogenic microorganism is generally unfavourable. The alpha diversity does not provide any indication as to the pathogenicity of the organisms but only the relative diversity.

To compare different microbiomes to each other, the beta diversity is used which is a measure of similarity or dissimilarity of two communities e.g., a comparison of the microbiomes of different hosts. The microbiome of a host may be evaluated using any method known to the skilled person, examples of such are 16s/18s and Internal Transcribed Spacer (ITS)analysis of the host microbiome, which in brief are sequencing methods that can distinguish different genus from each other based on the specific sequence of the 16s rRNA gene, 18s rRNA gene or ITS region sequence of specific microorganisms, such as fungi or bacteria.

Thus, in embodiments, a composition of the present invention may be for use in increasing the alpha diversity of a host microbiome following administration to said host. Furthermore, in embodiments, administration of a composition of the present invention to a host, enhances the alpha diversity of said host's bacterial microbiome. In further embodiments, the alpha diversity of a host's bacterial microbiome is evaluated by 16s, 18s and/or ITS sequencing.

In addition, in embodiments, a composition of the present invention may enhance or reduce the alpha diversity of a host fungal microbiome following administration to said host. In further embodiments, the alpha diversity of said host fungal microbiome is evaluated by 16s, 18s and/or ITS sequencing.

An enhanced or reduced alpha diversity of a host's microbiome is evaluated as a comparison to the basal host microbiome as measured before administration of a composition of the present invention.

In embodiments of the invention, the genetically engineered non-pathogenic microorganism expressing one or more polypeptides comprising the polypeptide hormone FGF19 or a functional homologue thereof, reduces the alpha diversity of a host fungal microbiome, following administration of said composition to beforementioned host.

In embodiments of the invention, the genetically engineered non-pathogenic microorganism expressing one or more polypeptides comprising the polypeptide hormone Aldafermin or a functional homologue thereof, reduces the alpha diversity of a host fungal microbiome, following administration of said composition to beforementioned host.

In addition, in embodiments of the invention, a genetically engineered non-pathogenic microorganism expressing one or more polypeptides comprising the polypeptide hormone IGF-1 or a functional homologue thereof, enhances the alpha diversity of a host fungal microbiome following administration of said composition to beforementioned host.

### Use in Modulation of Pathways

The present invention also relates to a use of the composition of the present invention in the modulation of one or more physiological pathways, such as but not limited to, thermogenesis, FoxO signalling pathway, glucagon signalling pathway, growth hormone synthesis secretion and action, pyruvate metabolism, insulin signalling pathway, insulin resistance, ErbB signaling pathway, sphingolipid signalling pathway MAPK signalling pathway, AMPK signalling pathway, mTOR signalling pathway, fatty acid biosynthesis, chemical carcinogenesis/reactive oxygen species and Citrate cycle (TCA cycle).

In example 4 of the present in disclosure, it was found that a targeted effect of E. Coli expressing FGF19 in combination with dietary change reduced the expression of specific genes involved in fatty acid uptake/transport, microsomal oxidation or peroxisomal beta oxidation, which are pathways known to be involved in metabolic liver diseases.

Thus, in embodiments, the composition of the present invention is for use in a subject for reducing the expression of genes, involved in metabolic liver diseases occurrence.

In further embodiments, the composition of the present invention is for use in a subject for reducing the expression of genes involved in fatty acid uptake/transport, microsomal oxidation and peroxisomal beta oxidation, such as but not limited to genes provide in table 3.

The term “reducing the expression of genes” is to be understood as genes that exhibit a reduced RNA level following administration of a composition according to the present invention, wherein the level is compared to administration of a non-hormone expressing cell, such as but not limited to administration of wild type E coli. Nissle 1917, such as is exemplified in example 4 of the present disclosure.

The RNA level of a transcribed gene may be measured as described in example 4 of the present disclosure, or in alternative ways well known to the skilled person.

In further embodiments, the composition of the present invention is for use in a subject for reducing the expression of one or more genes related to the Peroxisome proliferator-activated receptor (PPAR) signalling pathway.

One way to analyse changes in cellular pathways is, in example the KEEG enrichment analysis, as described in example 4. Changes in cellular pathways may also be analysed by other methods known to the skilled person.

In additional embodiments, the one or more genes related to the PPAR signalling pathway is/are selected from the group consisting of Pparg, Acaa1b, Cyp4a14, Slc27a1 and Cyp4a10.

In further embodiments, the composition of the present invention is for use in a subject for reducing the expression of one or more genes related to peroxisomes.

In further embodiments, the one or more genes related to peroxisomes is selected from the group consisting of Acaa1 b, Dhrs4, Crat, Hmgcl, Pex11a and Ech1.

In further embodiments, the composition of the present invention is for use in a subject for enhancing the expression of genes involved in PPAR signalling pathway and liver peroxisome, such as but not limited to genes provide in table 3.

The term “enhancing the expression of genes” is to be understood as genes that exhibit an enhanced RNA level following administration of a composition according to the present invention, wherein the level is compared to administration of a non-hormone expressing cell, such as but not limited to administration of wild type E. coli Nissle 1917, such as is exemplified in example 4 of the present disclosure.

In further embodiments, the composition of the present invention is for use in a subject for enhancing the expression of one or more genes related to the thermogenesis, FoxO signalling pathway, glucagon signalling pathway, growth hormone synthesis secretion and action, pyruvate metabolism, insulin signalling pathway, insulin resistance, ErbB signalling pathway, sphingolipid signalling pathway MAPK signalling pathway, AMPK signalling pathway, mTOR signalling pathway, fatty acid biosynthesis, chemical carcinogenesis/reactive oxygen species and/or Citrate cycle (TCA cycle).

In additional embodiments, the one or more genes related to the PPAR signalling pathway is/are selected from the group consisting of Pparg, Acaa1b, Cyp4a14, Slc27a1 and Cyp4a10.

In further embodiments, the composition of the present invention is for use in a subject for enhancing the expression of one or more genes related to the thermogenesis pathway.

In further embodiments, the one or more genes related to the thermogenesis pathway is selected from the group consisting of Acsl1, Sos1, Creb1, Ndufsl, Coa5, Atf2, Cpt2, Klb, Uqcrc2, Sos2, Cox18, Prkaal, ND1, ND2, COX1, COX2, ATP6, ND3, ND4, ND5, ND6, CYTB and ND4L.

In embodiments, the composition of the present invention is for use in a subject for enhancing one or more genes of the glucagon and/or insulin signalling pathway.

In embodiments, the composition of the present invention is for use in a subject for enhancing one or more genes involved in the regulation of glucose availability in adipocytes.

In further embodiments, the one or more genes involved in the regulation of glucose availability in adipocytes is selected from the group consisting of Sirt1, Pdhb, Pfkfbl, Phkg1, Creb1, Atf2, Phka2, Phkb, Ppp4r3a and Camk2d.

In embodiments, the composition of the present invention is for use in a subject for enhancing one or more genes of involved in the activation of lipogenesis in adipocytes.

In further embodiments, the one or more genes involved in the lipogenesis in adipocytes is selected from the group consisting of Acaca, Pdhb, Pdha1 and Acacb.

In embodiments, the composition of the present invention is for use in a subject for enhancing one or more genes of involved in proliferation and differentiation of adipocytes.

In further embodiments, the one or more genes involved in proliferation and differentiation of adipocytes is selected from the group consisting of Sos1, Sos2 and Mapk1.

TABLE 3
Genes following administration a composition of the invention.

| | | GenBank* | | | |
|---|---|---|---|---|---|
| | | Identifier | | | |
| | | (Mouse/ | | | GenBank |
| Gene | Related to | human) | Gene | Related to | Identifier |
| Pparg | PPAR signalling | 19016/5468 | Sirt1 | Glucose availability | 93759/23411 |
| Acaa1b | | 235674 | Pdhb | | 68263/5162 |
| Cyp4a14 | | 13119 | Pfkfb1 | | 18639/5207 |
| Slc27a1 | | 26457/376497 | Phkg1 | | 18682/5260 |
| Cyp4a10 | | 13117 | Creb1 | | 12912/1385 |
| Acaa1b | Peroxisomes | 235674 | Atf2 | | 11909/1386 |
| Dhrs4 | | 28200/10901 | Phka2 | | 110094/5256 |
| Crat | | 12908/1384 | Phkb | | 102093/5257 |
| Hmgcl | | 15356/3155 | Ppp4r3a | | 68734/55671 |
| Pex11a | | 18631/8800 | Camk2d | | 12324/817 |
| Ech1 | | 51798/1891 | Acaca | Lipogenesis | 107476/31 |
| Acsl1 | Thermogenesis | 14081/2180 | Pdhb | | 68263/5162 |
| Sos1 | | 20662/6654 | Pdha1 | | 18597/5160 |
| Creb1 | | 12912/1385 | Acacb | | 100705/32 |
| Ndufs1 | | 227197/4719 | Sos1 | Cellular proliferation | 20662/6654 |
| Coa5 | | 76187/493753 | Sos2 | and differentiation | 20663/6655 |
| Atf2 | | 11909/1386 | Mapk1 | | 26413/5594 |
| Cpt2 | | 12896/1376 | | | |
| Klb | | 83379/152831 | | | |
| Uqcrc2 | | 67003/7385 | | | |
| Sos2 | | 20663/6655 | | | |
| Cox18 | | 231430/285521 | | | |
| Prkaa1 | | 105787/5562 | | | |
| ND1 | | 5912287/4535 | | | |
| ND2 | | 3338894/4536 | | | |
| COX1 | | 5912286/4512 | | | |
| COX2 | | 5912281/4513 | | | |
| ATP6 | | 17705/4508 | | | |
| ND3 | | 5912285/4537 | | | |
| ND4 | | 17719/4538 | | | |
| ND5 | | 17721/4540 | | | |
| ND6 | | 17722/4541 | | | |
| CYTB | | 17711/4519 | | | |
| ND4L | | 17720/4539 | | | |

*GenBank ID may be retrived from: https://www.ncbi.nlm.nih.gov/gene/

### Use as a Medicament

The present invention also relates to a use of the composition of the present invention as a medicament. Thus, in an embodiment of the present invention, the composition is for use as a medicament.

In Example 1, it was shown that a host organism treated with a composition according to the present invention, in specific a composition comprising a genetically engineered non-pathogenic microorganism expressing one or more polypeptides, in specific Aldafermin or IGF-1, resulted in a weight loss of said host organism (Table 5).

Thus, in an embodiment the composition of the present invention is for use as a prophylactic. A further embodiment relates to the use of the composition of the present invention in reducing fat accumulation in the liver in a subject. Another embodiment relates to the use of the composition of the present invention for prophylactic treatment of subjects in risk of developing NAFLD or NASH. In embodiments the subjects in risk of developing NAFLD or NASH are subjects with a metabolic disease and/or syndrome.

### NAFLD and NASH

Non-alcoholic fatty liver disease (NAFLD) is a condition in which excess fat builds up in the liver. This build-up of fat is not caused by heavy alcohol use, but commonly a result of a high fat and/or high sugar diet.

Non-alcoholic fatty liver (NAFL) is a form of NAFLD in which the patients have fat in their liver but little or no inflammation or liver damage. NAFL typically does not progress to cause liver damage or complications. However, NAFL can cause pain from enlargement of the liver.

Nonalcoholic steatohepatitis (NASH) is the form of NAFLD in which the patients have inflammation of the liver and liver damage, in addition to fat in their liver. The inflammation and liver damage of NASH can cause fibrosis and/or scarring of the liver. NASH may lead to liver cirrhosis, in which the liver is scarred and permanently damaged. Cirrhosis can further lead to liver cancer.

In Example 2 and Example 3 it was showed that a host organism treated with a composition according to the present invention suffering from NAFLD have a reduced liver fat content, suggesting that a composition of the present invention is suitable for use as a treatment for NAFLD.

Thus, in embodiments a composition of the present invention is for use in the treatment of NAFLD and/or NASH. In embodiments a composition of the present invention is for use in the amelioration of NAFLD and/or NASH. In embodiments a composition of the present invention is for use in the reduction of the fat content in the liver of a subject. Thus, embodiments of the present invention provide methods for providing a composition for treatment of NAFLD to a subject in the need thereof.

In additional embodiments a composition comprising a genetically engineered non-pathogenic microorganism of the present invention is for use in the treatment of NAFLD. In additional embodiments a composition comprising a genetically engineered non-pathogenic microorganism of the present invention is for use in the treatment of NASH. In further embodiments, the composition of the present invention is administered by oral or rectal administration. In further embodiments, the composition of the present invention for use as a medicament is for oral administration. In other embodiment, the composition of the present invention is for administration by faecal microbiota transplantation. In that regard, in embodiments the composition of the present invention is provided as a tablet, capsule or suppository.

In an embodiment a genetically engineered non-pathogenic microorganism expressing one or more polypeptides, wherein said polypeptides comprise polypeptide hormones selected from the group consisting of FGF-19, Aldafermin, IGF-1 and functional homologues thereof, group consisting of FGF-19, Aldafermin, IGF-1 and functional homologues of FGF-19, Aldafermin and IGF-1, and wherein FGF-19 has an amino acid sequence identified by SEQ ID NO: 1, Aldafermin has an amino acid sequence identified by SEQ ID NO: 2 and IGF-1 has an amino acid sequence identified by SEQ ID NO: 3, and a functional homologue thereof has a sequence identity of at least 80%, such as 85%, such as 90%, such as 95% or such as at least 99% towards SEQ ID NO: 1, 2 or 3, is for use as a medicament.

In embodiments, the invention also provides a therapeutic regimen for modulating the peptide hormone homeostasis in NAFLD, wherein the dosage of the composition comprising the genetically engineered non-pathogenic microorganism may be determined on an individual basis, and the amount of the administrated microbiome-based therapeutic can be assessed and monitored on the basis of the individual patient's age, weight, food intake, macrobiotic flora, level of polypeptide hormones and glucose, as measured from e.g., blood samples, urine samples and/or faecal samples. Thus, in embodiments of the therapeutic regiment, the composition of the present invention is administered by oral and/or rectal administration or alternatively by fecal microbiota transplantation.

### Sequences

The current application contains a sequence listing in text format and electronical format which are hereby incorporated by reference.

An overview of the sequences is provided here, if there is discrepancy between the listed sequence and the public reference, the sequence in the sequence list prevails.

TABLE 4
Sequence overview

| | | SEQ |
|---|---|---|
| | | ID |
| Gene/protein | Description | NO |
| FGF-19 | Amino acid sequence of FGF-19 | 1 |
| Aldafermin | Amino acid sequence of Aldafermin | 2 |
| IGF-1 | Amino acid sequence of IGF-1 | 3 |
| FGF-19 | Nucleic acid sequence of FGF-19 | 4 |
| Aldafermin | Nucleic acid sequence of Aldafermin | 5 |
| IGF-1 | Nucleic acid sequence of IGF-1 | 6 |
| phoA | Signal peptide from E. Coli | 7 |
| lamB | Signal peptide from E. Coli | 8 |
| ompA | Signal peptide from E. Coli | 9 |
| fhuD | Signal peptide from E. Coli | 10 |
| mdoD | Signal peptide from E. Coli | 11 |
| ycdO | Signal peptide from E. Coli | 12 |
| dsbA | Signal peptide from E. Coli | 13 |
| usp45 | Signal peptide from L. lactis | 14 |
| eglA | Signal peptide from | |
| | C. saccharobutylicum | 15 |
| nprM3 | Signal peptide from C. sporogenes | 16 |
| alpha mating factor | Signal peptide S. boulardii | 17 |
| secretion signal | | |
| Schanzetta Promoter 8 | Artificial promoter sequence | 18 |
| Schanzetta Promoter 7 | Artificial promoter sequence | 19 |
| Schanzetta Promoter 6 | Artificial promoter sequence | 20 |
| J23107 | Promoter sequence from | 21 |
| TDH3 | Promoter sequence from S. cerevisiae | 22 |
| TEF1 | Promoter sequence from S. cerevisiae | 23 |

### EXAMPLES

### Method

### Preparation of a Composition for Administration

### Probiotic Preparation

Four different genetically engineered E. coli Nissle 1917 that continuously express exendin-4 (a GLP-1 analogue), adiponectin, aldafermin (engineered variant of the human FGF-19, SEQ ID NO: 2) and IGF-1 (SEQ ID NO: 3) was used. These hormonal analogues have demonstrated cross-reactivity with mice receptors similar to human ones.

To avoid repeated oral gavage which may induce potential injury and stress to mice, mice were trained to voluntarily consume gelatine cubes containing probiotics as adapted from the protocol developed by Zhang et al. (see Zhang, L., Method for voluntary oral administration of drugs in mice. STAR Protocols, 2021. 2(1):p. 100330). Probiotics were administered daily via gelatine cubes at the concentration of 109 CFU per cube. Probiotic gelatine cube stock of a total of five E. Coli Nissle 1917 strains were prepared including the control strain without hormone production (Group 2 and 9) and strains expressing IGF-1 (Group 3), GLP-1 (Group 4 and 10), FGF19 (Group 5), and Adiponectin (Group 6) respectively. Vehicle gelatine cubes were prepared without the probiotics for control groups (Group 1 and 8). The strains were grown overnight at 37° C. and 200 RPM in LB broth with 100 μg/ml Streptomycin+50 μg/ml Kanamycin. The next day, the cells were centrifugated at 10 min at 4000 RPM and the pellets were washed with 3 ml 0.9% saline solution twice. 100 μl of the overnight culture was then added to a LB agar plate (no antibiotics) and incubated at 37° C. overnight. The next day, the bacteria were harvested by adding 1 ml 10% Sweetener to the plate. 20% gelatine stock in sweetener was prepared and vanilla flavouring was added in 70:5 ratio. Lastly, 75 ul of the bacterial culture was mixed with 75 ul of the gelatine stock in a 96 well plate and the plate was stored in a 4 C fridge until probiotic administration. The next day, the concentration of 109 CFU per cube of viable E. Coli was confirmed by plating onto a LB agar plate with 50 μg/ml Streptomycin+50 μg/ml Kanamycin. During the probiotics administration, the mice were separated into individual cages and each mouse was provided with one gelatine cube to ensure every mice received the same dosage amount.

### Study Design

### Conducting the Animal Study

60 male JaxC57BL/6J (Charles River) mice at the age of 5- to 8-week-old were housed in the animal housing facilities at UEF and kept under specific-pathogen-free conditions in individually ventilated cages under a 12/12 h light cycle at 23° C. Mice were housed in groups of 2 to 4 and in the event of fighting between the mice and barbering issues, they were subsequently housed individually to avoid suffering. The mice were handled in accordance with Finnish legislation and the Council of European Convention ETS 123 on the use of vertebrate animals for scientific purposes. Mice were fasted for 2 hours (see Carper, D., et al., Reappraisal of the optimal fasting time for insulin tolerance tests in mice. Molecular metabolism, 2020. 42:p. 101058) before killed via terminal anaesthesia of Mebunat (pentobarbital) in accordance with the animal license. Depth of anaesthesia was controlled by pinching the rear leg with increasing dosing until the mouse was unresponsive which was followed by blood extraction by cardiac puncture, cardiac perfusion with 0.9% NaCl saline solution, death confirmation by cervical dislocation and lastly sample/tissue collection.

### Diet Intervention

The American Lifestyle-Induced Obesity Syndrome (ALIOS) diet used in our study (trans fat custom diet TD06303, Harlan Teklad, Madison, WI) is a high-fat diet adapted from the study by Tetri et al. (Tetri, L. H., et al., Severe NAFLD with hepatic necroinflammatory changes in mice fed trans fats and a high-fructose corn syrup equivalent. American Journal of Physiology-Gastrointestinal and Liver Physiology, 2008. 295(5): p. G987-G995) which mimics the western fast-food diet and induce NAFLD development in mice. The ALIOS diet has an energy density of 4.6 kcal/g and a fatty acid profile consisting of saturated (23%), trans (34%), monounsaturated (cis) (31%) and polyunsaturated (cis) (12%) fat which in total constituted 45% of the total energy and 23% of the weight of the diet. In addition to the ALIOS diet, ALIOS mice were given high-fructose corn syrup (HFCS) equivalent (45% glucose and 55% fructose by weight) at 42 g/I in their drinking water. The ALIOS diet and high-fructose corn syrup equivalent were used to induce NAFLD development in the mice (see Tetri, L. H., et al., Severe NAFLD with hepatic necroinflammatory changes in mice fed trans fats and a high-fructose corn syrup equivalent. American Journal of Physiology-Gastrointestinal and Liver Physiology, 2008. 295(5): p. G987-G995). A pilot study conducted by our group have demonstrated that C57BL/6 mouse develops NAFLD after 12 to 16 weeks of American Lifestyle-Induced Obesity Syndrome (ALIOS) diet. Control mice were fed the standard rodent chow control diet (2016 Teklad global 16% protein rodent diets, Harlan Teklad, Madison, WI) which has an energy density of 3 kcal/g and plain water without HFCS.

60 mice were randomly assigned into 10 groups (6 mice per group). They were fed ad libitum either the American Lifestyle-Induced Obesity Syndrome (ALIOS) diet (Groups 1 to 7: 42 mice) or the standard rodent chow control diet (Groups 8 to 10: 18 mice) for 14 weeks (see FIG. 8). One mouse from Group 7 died due to unknown causes and was excluded from the study.

### Lifestyle Modification and Probiotic Intervention

Following the diet intervention, all the ALIOS mice were switched to the standard rodent chow control diet during the 7-week lifestyle modification intervention period. ALIOS mice in Group 1 and Control mice in Group 8 were control groups where the mice were not given the probiotic treatment and were given vehicle gelatine cubes. ALIOS mice in Group 2 and Control mice in Group 9 were control groups for the hormonal intervention where the conventional E. Coli Nissle 1917 strain given did not produce any hormone. ALIOS mice in Groups 3 to 6 underwent probiotic intervention where the administered engineered E. Coli Nissle 1917 strains expressed 4 different hormones (Group 3: IGF1; Group 4: GLP-1; Group 5: FGF19; Group 6: Adiponectin) respectively. Group 7 was the positive control group where Liraglutide is a GLP-1 receptor agonist used for the treatment of metabolic diseases such as type 2 diabetes and obesity. Control mice in Group 10 underwent probiotic intervention where the administered engineered E. Coli Nissle 1917 strains expressed GLP-1 which served as a control for testing, in non-NAFLD conditions, the safety of consuming genetically modified E. coli Nissle 1917 expressing exendin-4. Weight was weekly monitored (daily for the group 7 to adjust liraglutide dosage). Fecal samples was collected daily for the first two weeks in all groups and the day before sacrifice in Week 7.

### Liraglutide Intervention

Group 7 was the positive control groups where Liraglutide was administered daily at 0.2 mg/kg body weight via subcutaneous injection during the 7-week intervention period.

### Example 1

### Materials and Methods

Body weight was measured weekly throughout the intervention period. The body weight loss was calculated by the difference between the final and initial body weight during the 7 weeks of lifestyle modification intervention (table 5).

### Results

Due to high variability of body weight of the mice between the groups at the baseline before the lifestyle modification intervention, absolute body weight loss is more appropriate for measuring the effect of different treatments on the mice by normalizing baseline differences. Results from absolute body weight loss showed that Group 3 (E. coli+IGF) and 5 (E. coli+FGF19) ALIOS mice had consistently significant weight loss compared with Group 8 to 10 Control mice (see table 5).

The body weight loss of Group 3 (E. coli+IGF) and 5 (E. coli+FGF19) ALIOS mice showed low variability within the groups while in Group 1 (ALIOS mice control) and Group 2 (E. coli control) ALIOS mice had high variability. Therefore, even though, Group 3 (E. coli+IGF) and 5 (E. coli+FGF19) ALIOS mice did not show statistically significant increase in body weight loss compared to Group 1 (ALIOS mice control) and Group 2 (E. coli control) ALIOS mice, we observed a clear trend of difference in body weight loss (see table 5).

TABLE 5

| | Group | | | | | | | | | |
|---|---|---|---|---|---|---|---|---|---|---|
| | | | | 4 | 5 | | | | | 10 |
| | | 2 | 3 | ALIOS | ALIOS | 6 | 7 | | 9 | Control |
| | 1 | ALIOS | ALIOS | E. coli | E. coli | ALIOS | ALIOS | 8 | Control | E. coli |
| | ALIOS | E. coli | E. coli | Nissle | Nissle | E. coli | Liraglutide | Control | E. coli | Nissle |
| | diet | Nissle | Nissle | (GLP-1) | (FGF19) | Nissle | (Positive | diet | Nissle | (GLP-1) |
| | (control) | (control) | (IGF1) | (Exendin-4) | (Aldafermin) | (Adiponectin) | control) | (control) | (control) | (Exendin-4) |
| Weight | 0.05 ± | 0.25 ± | 1.783 ± | 1.167 ± | 2.217 ± | 0.6667 ± | 0.38 ± | −1.383 ± | −1.667 ± | −1.25 ± |
| loss (g) | 2.101^{a,b,c} | 1.159^{a,b,c} | 0.5845^{a} | 2.145^{a,b} | 0.5913^{a} | 1.573^{a,b,c} | 1.137^{a,b,c} | 0.8819^{b,c} | 0.689^{c} | 1.601^{b,c} |
| MRI fat | 12.68 ± | 21.97 ± | 11.87 ± | 6.7 ± | 10.84 ± | 12 ± | 9.543 ± | 5.501 ± | 8.46 ± | 6.3 ± |
| index | 6.235 | 7.241 | 4.464 | 3.175 | 2.149 | 7.597 | 3.809 | 0.5728 | 6.548 | 0.02828 |
| (Week 0) | | | | | | | | | | |
| MRI fat | 8.067 ± | 7.973 ± | 3.684 ± | 3.426 ± | 3.451 ± | 4.259 ± | 4.702 ± | 5.043 ± | 3.748 ± | 3.821 ± |
| index | 4.953 | 1.584 | 0.4426 | 0.645 | 0.3377 | 1.376 | 0.1535 | 1.135 | 1.939 | 0.594 |
| (Week 7) | | | | | | | | | | |
| Decrease | 4.614 ± | 14 ± | 8.182 ± | 3.274 ± | 7.393 ± | 7.741 ± | 4.842 ± | 0.4573 ± | 3.985 ± | 2.743 ± |
| in MRI | 3.453 | 6.582 | 4.062 | 2.533 | 2.173 | 6.373 | 3.739 | 0.5844 | 4.463 | 0.5636 |
| fat index | | | | | | | | | | |

Data shown as mean ± SD.
Superscript letters (^{a, b, c}) showed the statistically significant differences amongst the groups.
Groups with the same letters had no statistically significant differences (p < 0.05).
For Weight loss (g), (Group 1-6, and 8-10: n = 6 per group; Group 7: n = 5).
The body weight loss was calculated by the difference between the final and initial body weight during the 7 weeks of lifestyle modification intervention.
Decrease in MRI fat index was calculated by the difference between the final and initial MRI fat index during the 7 weeks of lifestyle modification intervention.
Analysis done with Graphpad, Oneway-ANOCA, Tukey's multiple comparisons test.
For MRI fat index, (Group 1: n = 5; Group 2 to 8: n = 3 per group; Group 9-10: n = 2 per group at Lifestyle 0 and n = 3 per group at Lifestyle 7).

Table 5 shows absolute body weight loss of mice after 7 weeks of lifestyle modification (FIG. 4) intervention and MRI liver fat index measured before (Week 0) and after (Week 7) the 7 weeks of lifestyle modification intervention.

### Example 2—MRI Imaging and Liver Fat Content

### Material and Methods

MRI was used before the start and in the last week of the lifestyle modification intervention, which is a non-invasive analysis of morphology and fat content in liver. Results are expressed as fat index which is the ratio of fat to water content in the liver to measure the extent of fat accumulation in the liver.

### Results

After 7 weeks of the lifestyle modification intervention, the ALIOS mice overall had a greater extent of reduction in lipid accumulation in the liver than the control groups and the variabilities within the groups were decreased (see table 5).

### Example 3—Liver Oil Red O and Haematoxylin Eosin Staining

### Materials and Methods

To assess the effect of the diet and the impact of probiotic in the liver, a quantitative method for measuring the liver steatosis by Oil Red-O (ORO) staining and digital image analysis is used (see Arganda-Carreras, I., et al., Trainable Weka Segmentation: a machine learning tool for microscopy pixel classification. Bioinformatics, 2017. 33(15): p. 2424-2426; Levene, A. P., et al., Quantifying hepatic steatosis-more than meets the eye. Histopathology, 2012. 60(6):p. 971-981 Moreover, ORO staining and digital image analysis of liver steatosis well correlate with the total liver triglyceride concentration and increase the diagnostic accuracy for identifying lipids providing a more accurate quantification of microvesicular and macrovesicular steatosis (see Levene, A. P., et al., Quantifying hepatic steatosis—more than meets the eye. Histopathology, 2012. 60(6):p. 971-981). In addition, the metabolic status in health and disease can be evaluated quantifying hepatic lipid accumulation as described by Mehlem et al. (Mehlem, A., et al., Imaging of neutral lipids by oil red 0 for analysing the metabolic status in health and disease. Nature protocols, 2013. 8(6): p. 1149-1154). In addition, visual observations by ORO staining were further confirmed by Haematoxylin Eosin (H&E) staining.

### Results

Histological figures with ORO staining (see FIG. 1A) of the left lateral lobe of mouse liver showed that ALIOS mice Group 3 (E. coli+IGF1), 4 (E. coli+GLP-1) and 5 (E. coli+FGF19) did not show fat droplets in the liver while ALIOS mice Group 1 (ALIOS control), 2 (E. coli control) and 7 (Liraglutide) and Control diet mice Group 8 (Control diet only) and 9 (E. coli+GLP-1), had few fat droplets in the liver. Quantification of the liver fat droplets percentage from the ORO staining (see FIG. 2) showed that ALIOS mice Group 3 (E. coli+IGF1), 4 (E. coli+GLP-1), 5 (E. coli+FGF19) have significantly lower percentage of liver fat droplets compared with Group 1 (ALIOS control). ORO staining visual observations were also confirmed by H&E staining (see FIG. 1B).

### Example 4—RNA-Sequencing Analysis in Liver and Epididymal Adipose Tissue

### Materials and Methods

Transcriptomics analysis of mRNA were performed using NovaSeq 6000 sequencing platform. Strategy by Novogene Co., Ltd., from liver and epididymal adipose tissues samples in ALIOS mice group 5 (E. coli+FGF19), ALIOS mice Group 1 (ALIOS control), 2 (E. coli control), 7 (Liraglutide), Control diet mice Group 8 (Control diet only) and 9 (E. coli control) was adopted to investigate the gene expression profiles. Differential expression analysis for comparisons among samples (five biological replicates per condition) was performed using DESeq2 R package (Love M I. et al., (2014), “Moderated estimation of fold change and dispersion for RNA-seq data with DESeq2.” Genome Biology, 15, 550) The resulting P values were adjusted using the Benjamini and Hochberg's approach for controlling the False Discovery Rate (FDR). Genes with an adjusted P value<0.05 found by DESeq2 were assigned as differentially expressed. KEEG enrichment analysis was performed for understanding high-level functions of significantly enriched differential expressed genes and implemented by clusterProfiler R package (Wu T. et al., (2021). clusterProfiler 4.0: A universal enrichment tool for interpreting omics data. The Innovation, 2(3), 100141; Yu G. et al., 2011, clusterProfiler: an R package for comparing biological themes among gene clusters. OMICS: A Journal of Integrative Biology, 16(5), 284-287) in which gene length bias was corrected. KEEG pathways with Pvalue adjusted using Benjamini and Hochberg's approach (for controlling the FDR)<0.05 were considered significantly enriched by differential expressed genes.

### Results

After 7 weeks of lifestyle interventions liver and epididymal adipose tissue showed clear different gene expression profiles and three of five replicates belonging to Group 5 ALIOS+E.Coli FGF19 showed a clear cluster while two of them were separated in both liver and epididymal adipose tissue (see FIG. 3). RNA sequencing analysis showed that after 7 weeks of intervention, Group 5 (ALIOS+E.Coli FGF19) induced significant transcriptomic changes in liver and epididymal adipose tissue compared to ALIOS mice Group 1 (ALIOS control) and 2 (E. coli control) (see FIG. 4) showing that relevant change in gene expression due to the targeted effect of FGF19 in liver and epididymal adipose tissue.

Further KEEG enrichment analysis showed clearly the potential effect of E.Coli FGF19 in combination with dietary change in downregulation of PPAR signaling pathway and peroxisome in liver (see FIG. 5). Of regard of PPAR signaling pathway, in Group 5 (ALIOS+E.Coli FGF19) we observed downregulation of genes Pparg, Plin4, Plin2, Cyp4a14, Slc27a1, Acadm and Cyp4a10 compared to Group 1 (ALIOS control) and genes Pparg, Acaa1 b, Cyp4a14, Slc27a1 and Cyp4a10 compared to Group 2 (E. coli control); While for peroxisome pathway we reported downregulation of genes Crot, Pex7, Dhrs4, Hmgcl and Ech1 compared to Group 1 (ALIOS control) and genes Acaa1 b, Dhrs4, Crat, Hmgcl, Pex11a and Ech1 compared to Group 2 (E. coli control). This finding showed a targeted potential effect of E.Coli FGF19 in combination with dietary change to at least partially reduce the expression of genes involved in fatty acid uptake/transport, microsomal oxidation and peroxisomal beta oxidation which are involved in metabolic liver diseases occurrence (Koonen D P et. al., 2007, increased hepatic CD36 expression contributes to dyslipidemia associated with diet-induced obesity. Diabetes. 2007; 56:2863-2871; Skat-Rordam et al., 2019, A role of peroxisome proliferator-activated receptor γ in non-alcoholic fatty liver disease, Basic Clin Pharmacol Toxicol. 124(5):528-537; Zhang X., et al., Ablation of cytochrome P450 omega-hydroxylase 4A14 gene attenuates hepatic steatosis and fibrosis, Biological Sciences, 114 (12) 3181-3185; Leclercq I A, et al. CYP2E1 and CYP4A as microsomal catalysts of lipid peroxides in murine nonalcoholic steatohepatitis. J Clin Invest. 2000; 105(8):1067-1075; Ipsen D H., et al., 2018, Molecular mechanisms of hepatic lipid accumulation in non-alcoholic fatty liver disease, Cell. Mol. Life Sci., 75, pages 3313-3327)), and this is in line with ORO staining results in which we showed a reduction of liver steatosis after the use of E.Coli FGF19 compared to Group 1 (ALIOS control) and Group 2 (E. coli control).

KEEG enrichment analysis have been performed also in epididymal adipose tissue (see FIG. 6) and the common upregulated pathways in Group 5 (ALIOS+E.Coli FGF19) compared to both Group 1 (ALIOS control) and Group 2 (E. coli control) were the Thermogenesis, FoxO signaling pathway, Glucagon signaling pathway, Growth hormone synthesis secretion and action, Pyruvate metabolism, Insulin signaling pathway, Insulin resistance, ErbB signaling pathway, Sphingolipid signaling pathway MAPK signaling pathway, AMPK signaling pathway, mTOR signaling pathway, Fatty acid biosynthesis, Chemical carcinogenesis/reactive oxygen species and Citrate cycle (TCA cycle). In the thermogenesis pathways, Group 5 (ALIOS+E.Coli FGF19) showed upregulation of genes Prkacb, Acsl1, Frs2, Sos1, Creb1, Adcy10, Prkaa2, Uqcrc2, Acs14, Sos2, Prdm16, Cox17, Kdm3a, COX1, ND3, ND4, ND5, CYTB and Cox16 compared to Group 1 (ALIOS control) while Acsl1, Sos1, Creb1, Ndufsl, Coa5, Atf2, Cpt2, Klb, Uqcrc2, Sos2, Cox18, Prkaal, ND1, ND2, COX1, COX2, ATP6, ND3, ND4, ND5, ND6, CYTB and ND4L compared to Group 2 (E. coli control), showing a targeted positive effect in genes involved in stimulation of the browning of white adipose tissue which have been showed to confer beneficial effects on adiposity (Bartelt, A., Heeren, J. Adipose tissue browning and metabolic health. Nat Rev Endocrinol 10, 24-36 (2014).

In addition, in thermogenesis pathway we observed upregulation of Frs2 and KLb implicated in FGF19 subfamily signaling (Dolegowska K. et al., 2019, FGF19 subfamily members: FGF19 and FGF21, J. Physiol. Biochem, 75, pages 229-240). Moreover, we observed upregulation of both glucagon and insulin signaling pathway after 7 weeks of probiotic FGF19 intervention compared to Group 1 and 2; in specific we showed upregulation of genes linked to the increase of glucose availability in adipocytes (Sirt1, Ppara, Creb1, Phkb, Ppp4r3a compared to Group 1 and Sirt1, Pdhb, Pfkfbl, Phkg1, Creb1, Atf2, Phka2, Phkb, Ppp4r3a, Camk2d compared to Group 2), genes linked to activation of lipogenesis in adipocytes (Acaca, Pdhb, Pdha1, Phkb, Acacb, FASN compared to Group 1 and Acaca, Pdhb, Pdha1, Acacb compared to Group 2) and genes linked to proliferation and differentiation of adipocytes (Sos1, Sos2, Mapk1 compared to both Group 1 and 2).

Overall, our RNA-sequencing results showed how the probiotic E. Coli FGF19 is able to induce metabolic improvements in adipose in liver and epididymal adipose tissue axis.

### Example 5—Bacterial Gut Microbiota 16s Analysis

### Materials and Methods

Metagenomics 16s ribosomal RNA sequencing were performed using NovaSeq 6000 sequencing platform and strategy by Novogene Co., Ltd., from cecum content samples to investigate the bacterial composition.

### Results

ANOVA analysis of the observed microbial richness (see FIG. 7) showed that the different groups have significantly different alpha diversity (p-value: 0.011123; F-value: 3.3183) where ALIOS mice Group 3 (E. coli+IGF1) have significantly higher microbial richness compared with Group 1 (ALIOS control). PERMANOVA analysis of the beta-diversity (Distance method: Jensen-Shannon Divergence) showed that there are statistically significant differences in the microbial community composition among the different treatment groups in ALIOS mice (F-value: 6.2158; R-squared: 0.52311; p-value<0.001). Further heat tree analysis of significant taxonomic differences (Wilcoxon p-value<0.05) between microbial communities showed that compared with Group 1 (ALIOS control), Group 3 (E. coli+IGF1) had lower abundance of the phylum Desulfobacterota, in particular in the genus Bilophila. These microorganisms are sulfate-reducing bacteria which are associated with gut inflammation (Braccia, D. J., et al., (2021) The capacity to produce hydrogen sulfide (H2S) via cysteine degradation is ubiquitous in the human gut microbiome. Frontiers in microbiology, 12; Figliuolo, V. R., et al., (2017) Sulfate-reducing bacteria stimulate gut immune responses and contribute to inflammation in experimental colitis. Life sciences, 189, 29-38). In addition, Group 3 had higher abundance of Roseburia (a butyrate producing bacteria (Takahashi, K., et al., (2016) Reduced abundance of butyrate-producing bacteria species in the fecal microbial community in Crohn's disease. Digestion, 93(1), 59-65). Group 5 (E. coli+FGF19) had lower abundance of the class Coriobacteriia and Acetatifactor muris, both are associated with intestinal inflammation (Alam, M. T., et al., (2020). Microbial imbalance in inflammatory bowel disease patients at different taxonomic levels. Gut pathogens, 12(1), 1-8; Yusufu, I., et al., (2021). A tryptophan-deficient diet induces gut microbiota dysbiosis and increases systemic inflammation in aged mice. International journal of molecular sciences, 22(9), 5005); while having higher abundance of Roseburia.

### ITEMS

1. A composition comprising a genetically engineered non-pathogenic microorganism expressing one or more polypeptides, wherein said polypeptides comprise polypeptide hormones selected from the group consisting of FGF-19, Aldafermin, IGF-1 and functional homologues of FGF-19, Aldafermin and IGF-1, and wherein FGF-19 has an amino acid sequence identified by SEQ ID NO: 1, Aldafermin has an amino acid sequence identified by SEQ ID NO: 2 and IGF-1 has an amino acid sequence identified by SEQ ID NO: 3, and a functional homologue thereof has a sequence identity of at least 80%, such as 85%, such as 90%, such as 95% or such as at least 99% towards SEQ ID NO: 1, 2 or 3.

2. The composition according to item 1, wherein said polypeptides are fusion peptide, chimeric peptide, and/or oligomeric peptide variants and/or combinations, of FGF-19, Aldafermin and IGF-1.

3. The composition according to items 1 or 2, wherein said polypeptide comprises an N-terminal polypeptide, such as a signal peptide, selected from the group consisting of polypeptides with an amino acid sequence according to SEQ ID NOs: 7, 8, 9, 10, 11, 12, 13, 14, 15, 16 and 17, in which said N-terminal polypeptide promotes excretion of said one or more polypeptides from said genetically engineered non-pathogenic microorganism.

4. The composition according to any of items 1-3, wherein said polypeptide is FGF-19 with an amino acid sequence identified by SEQ ID NO: 1, or a functional homologue thereof with a sequence identity of at least 80%, such as 85%, such as 90%, such as 95% or such as at least 99% towards SEQ ID NO: 1.

5. The composition according to any of items 1-3, wherein said polypeptide is Aldafermin, with an amino acid sequence identified by SEQ ID NO: 2, or a functional homologue thereof with a sequence identity of at least 80%, such as 85%, such as 90%, such as 95% or such as at least 99% towards SEQ ID NO: 2.

6. The composition according to any of items 1-3, wherein said polypeptide is IGF-1, with an amino acid sequence identified by SEQ ID NO: 3, or a functional homologue thereof with a sequence identity of at least 80%, such as 85%, such as 90%, such as 95% or such as at least 99% towards SEQ ID NO: 3.

7. The composition according to any of items 2-3, wherein said polypeptide is a fusion polypeptide comprising Aldafermin and IGF-1, wherein the fusion polypeptide comprises the amino acid sequence identified by SEQ ID NO: 2 and 3 or functional homologues thereof with a sequence identity of at least 80%, such as 85%, such as 90%, such as 95% or such as at least 99% towards Aldafermin of SEQ ID NO: 2 or IGF-1 of SEQ ID NO: 3.

8. The composition according to any of items 2-3, wherein said polypeptide is a fusion polypeptide comprising two or more repetitions of FGF-19, Aldafermin or IGF-1, wherein FGF-19 has an amino acid sequence identified by SEQ ID NO: 1, Aldafermin has an amino acid sequence identified by SEQ ID NO: 2 and IGF-1 has an amino acid sequence identified by SEQ ID NO: 3, and a functional homologue thereof has a sequence identity of at least 80%, such as 85%, such as 90%, such as 95% or such as at least 99% towards SEQ ID NO: 1, 2 or 3.

9. The composition according to any of items 2-3, wherein said polypeptide is a chimeric polypeptide comprising FGF-19, Aldafermin and/or IGF-1, wherein FGF-19 has an amino acid sequence identified by SEQ ID NO: 1, Aldafermin has an amino acid sequence identified by SEQ ID NO: 2 and IGF-1 has an amino acid sequence identified by SEQ ID NO: 3, and a functional homologue thereof has a sequence identity of at least 80%, such as 85%, such as 90%, such as 95% or such as at least 99% towards SEQ ID NO: 1, 2 or 3.

10. The composition according to any of the preceding items, wherein the microorganism is a yeast or bacteria.

11. The composition according to any of the preceding items, wherein the microorganism is of a genus selected from of the group consisting of Escherichia, Bacteroides, Clostridiales, Bifidobacteriales, Eubacteriales and Lactobacillales.

12. The composition according to any of the preceding items, wherein the microorganism is a species group consisting of Escherichia coli, Anaerobutyricum soehngenii, Lactococcus lactis, Bacteroides thetaiotaomicron, Bacteroides oavtus, Bacteroides xylanisolvens, Clostridium symbiosum and Saccharomyces bourladii.

13. The composition according to any of the preceding items, wherein the microorganism is selected from the group consisting of Escherichia coli Nissle 1917, Escherichia coli Symbioflor G1/2, Escherichia coli Symbioflor G4/9, Escherichia coli Symbioflor G5, Escherichia coli Symbioflor G6/7, Escherichia coli Symbioflor G, Anaerobutyricum soehngenii L2-7, Lactococcus lactis mg1363 and Saccharomyces bourladii MYA-796

14. The composition according to any of the preceding items, wherein the microorganism is Escherichia Coli.

15. The composition according to any of the preceding items, wherein the microorganism is Escherichia Coli Nissle 1917.

16. The composition according to any of the preceding items, wherein the microorganism comprises a recombinant nucleic acid sequence of SEQ ID NO: 4, 5 and/or 6, or a homologue thereof with a sequence identity of at least 70%, such as 80%, such as 90%, such as 95% or such as at least 99% towards SEQ ID NO: 4, 5 or 6, or the reverse complement thereof, encoding said polypeptides as defined in claim 1.

17. The composition according to item 16, wherein the recombinant nucleic acid further comprises one or more promoter elements for modulating the expression of said one or more polypeptides.

18. The composition according to item 17, wherein said promoter element, is selected from the group consisting of promoter sequences of SEQ ID NOs: 18, 19, 20, 21, 22 and 23.

19. The composition according to any of items 17-18, wherein said promoter sequence comprises a nucleic acid sequence according to SEQ ID NO: 18.

20. The composition according to any of items 16-19, wherein the recombinant nucleic acid sequence is comprised in an expression vector.

21. The composition according to any of items 16-20, wherein the recombinant nuclei acid sequence or expression vector is integrated into the genome of the genetically engineered non-pathogenic microorganism.

22. The composition according to any of items 16-20, wherein the recombinant nuclei acid sequence or expression vector is integrated into a plasmid.

23. The composition according to item 22, wherein the plasmid is an endogenous, exogeneous or artificial plasmid.

24. The composition according to any of items 22-23, wherein the plasmid is an endogenous plasmid.

25. The composition according to any of items 22-24, wherein the plasmid is endogenous E. coli plasmid pMUT1.

26. The composition according to any of items 22-25, wherein the plasmid is integrated into the genetically engineered non-pathogenic microorganism by transformation.

27. A composition as defined in any of the preceding claims for use as a medicament.

28. The composition for use according to item 27 in the treatment of NAFLD and/or NASH by oral administration.

29. The use according to any of items 27-28, in the treatment of NAFLD by oral administration.

1. A composition comprising a genetically engineered non-pathogenic microorganism expressing one or more polypeptides, wherein said polypeptides comprise polypeptide hormones selected from the group consisting of FGF-19, Aldafermin, IGF-1 and functional homologues of FGF-19, Aldafermin and IGF-1, and wherein FGF-19 has an amino acid sequence identified by SEQ ID NO: 1, Aldafermin has an amino acid sequence identified by SEQ ID NO: 2 and IGF-1 has an amino acid sequence identified by SEQ ID NO: 3, and a functional homologue thereof has a sequence identity of at least 80%, such as 85%, such as 90%, such as 95% or such as at least 99% towards SEQ ID NO: 1, 2 or 3.

2. The composition according to claim 1, wherein said polypeptides are fusion peptide, chimeric peptide, and/or oligomeric peptide variants and/or combinations, of FGF-19, Aldafermin and IGF-1.

3. The composition according to claim 1, wherein said polypeptide comprises an N-terminal polypeptide, selected from the group consisting of polypeptides with an amino acid sequence according to SEQ ID NOs: 7, 8, 9, 10, 11, 12, 13, 14, 15, 16 and 17, in which said N-terminal polypeptide promotes excretion of said one or more polypeptides from said genetically engineered non-pathogenic microorganism.

4. The composition according to claim 1, wherein the microorganism is of a genus selected from of the group consisting of Escherichia, Bacteroides, Clostridiales, Bifidobacteriales, Eubacteriales and Lactobacillales.

5. The composition according to claim 1, wherein the microorganism is Escherichia coli.

6. The composition according to claim 1, wherein the microorganism comprises a recombinant nucleic acid sequence according to SEQ ID NO: 4, 5 and/or 6, or a homologue thereof with a sequence identity of at least 70% towards SEQ ID NO: 4, 5 or 6 or the reverse complement thereof, encoding said polypeptides as defined in claim 1.

7. The composition according to claim 6, wherein the recombinant nucleic acid further comprises one or more promoter elements for modulating the expression of said one or more polypeptides.

8. The composition according to claim 7, wherein said promoter sequence comprises a nucleic acid sequence according to SEQ ID NO: 18.

9. The composition according to claim 6, wherein the recombinant nucleic acid sequence is comprised in an expression vector.

10. The composition according to claim 6, wherein the recombinant nuclei acid sequence or expression vector is integrated into the genome of the genetically engineered non-pathogenic microorganism.

11. The composition according to claim 6, wherein the recombinant nuclei acid sequence or expression vector is integrated into an endogenous, exogeneous or artificial plasmid.

12. The composition according to any of claim 11, wherein the plasmid is endogenous E. coli plasmid pMUT1.

13. (canceled)

14. (canceled)

15. (canceled)

16. A method for treating an individual suffering from NAFLD and/or NASH, the method comprising administering a therapeutically effective amount of the composition of claim 1.

17. The method of claim 16, wherein the administering is by oral administration.


SEQUENCE LISTING (23 sequences)

SEQ ID NO: 1 - protein, 192 aa, Homo sapiens
LAFSDAGPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 2 - protein, 191 aa, Homo sapiens
AMRDSSPLVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 3 - protein, 70 aa, Homo sapiens
GPETLCGAELVDALQFVCGDRGFYFNKPTGYGSSSRRAPQTGIVDECCFRSCDLRRLEMYCAPLKPAKSA

SEQ ID NO: 4 - DNA, 516 bp, synthetic construct
  source 1..516: mol_type = other DNA; note = synthetic nucleotide sequence encoding FGF-19
cgtcatctttacacgtcagggccccacggattgtcatcgtgctttcttcgcattcgtgccgatggtgttgttgactgtgctcgcggccagagtgcacatagcttgttagaaattaaagcggttgcactgcgcacagtagcgattaagggagttcactctgttcgttatctttgcatgggagccgacggaaaaatgcagggtctgttgcaatactccgaagaggactgtgccttcgaggaagagatccgcccagacgggtacaatgtgtatcgcagcgaaaagcaccgtcttcccgtttctttatcctctgctaaacaacgtcagttatataaaaaccgtggatttcttccgctgagtcattttttacccatgttaccaatggttcccgaagagccagaggacttgcgtgggcatttggaaagcgacatgttttcgtcgcctcttgaaacagactctatggatccattcggcttagtcacgggcctggaagccgttcgctcaccctccttcgagaag

SEQ ID NO: 5 - DNA, 570 bp, synthetic construct
  source 1..570: mol_type = other DNA; note = synthetic nucleotide sequence encoding Aldafermin
atgcgcgactcaagtccacttgtccattatgggtggggagatccgattcgtttacgccacttgtataccagtgggccacacggtttgtcatcctgtttccttcgtatccgtgccgacggagtggtcgactgtgcccgcggtcaatctgcccacagcttattagaaattaaagcggtcgctctgcgtacagtcgccattaagggagtccattctgttcgctacttgtgcatgggtgccgatggcaagatgcaggggcttttacaatactcggaggaagactgtgcattcgaggaagagattcgtcccgatggatacaacgtctaccgttcggagaagcaccgccttccggtaagtttgtcatccgcgaagcagcgtcagttgtataaaaatcgcggcttcctgcccttatctcatttccttccgatgcttcctatggtaccagaggaaccagaggatttgcgcggccaccttgagtccgatatgtttagttctcccttggaaactgattcaatggaccccttcggcctggtgactgggttggaggctgttcgctcgccgtcatttgagaaa

SEQ ID NO: 6 - DNA, 210 bp, synthetic construct
  source 1..210: mol_type = other DNA; note = synthetic nucleotide sequence encoding IGF-1
ggcccagagactttgtgtggagccgagttagtcgacgccttgcaattcgtatgcggagatcgtggcttctactttaacaagcctacaggatacggctcttcttcacgccgcgctccgcaaacaggcattgtagatgaatgctgttttcgctcctgtgacctgcgccgcctggaaatgtattgtgccccactgaaacctgcgaagagcgca

SEQ ID NO: 7 - protein, 21 aa, Escherichia coli
MKQSTIALALLPLLFTPVTKA

SEQ ID NO: 8 - protein, 25 aa, Escherichia coli
MMITLRKLPLAVAVAAGVMSAQAMA

SEQ ID NO: 9 - protein, 21 aa, Escherichia coli
MKKTAIAIAVALAGFATVAQA

SEQ ID NO: 10 - protein, 30 aa, Escherichia coli
MSGLPLISRRRLLTAMALSPLLWQMNTAHA

SEQ ID NO: 11 - protein, 32 aa, Escherichia coli
MDRRRFIKGSMAMAAVCGTSGIASLFSQAAFA

SEQ ID NO: 12 - protein, 26 aa, Escherichia coli
MTINFRRNALQLSVAALFSSAFMANA

SEQ ID NO: 13 - protein, 19 aa, Escherichia coli
MKKIWLALAGLVLAFSASA

SEQ ID NO: 14 - protein, 27 aa, Lactococcus lactis
MKKKIISAILMSTVILSAAAPLSGVYA

SEQ ID NO: 15 - protein, 38 aa, Clostridium saccharobutylicum
MFSKIKKINFFKKTFSFLIAVVMMLFTVLGTNTYKAEA

SEQ ID NO: 16 - protein, 25 aa, Clostridium sporogenes
MKSKKLLATVLSAVITLSTVSAVYA

SEQ ID NO: 17 - protein, 91 aa, Saccharomyces boulardii
MRFPSIFTAVLFAASSALAAPVNTTTEDETAQIPAEAVIGYSDLEGDFDVAVLPFSNSTNNGLLFINTTIASIAAKEEGVSLDKREEGEPK

SEQ ID NO: 18 - DNA, 36 bp, synthetic construct
  source 1..36: mol_type = other DNA; note = synthetic promoter sequence
tgcttgactcgtcgttatcctacgtgtataattggc

SEQ ID NO: 19 - DNA, 36 bp, synthetic construct
  source 1..36: mol_type = other DNA; note = synthetic promoter sequence
tgcttgactcgtcgttcctcctacgtgtataattgg

SEQ ID NO: 20 - DNA, 36 bp, synthetic construct
  source 1..36: mol_type = other DNA; note = synthetic promoter sequence
tgctggactcgtcgtaatcctgcgtgtataattggc

SEQ ID NO: 21 - DNA, 35 bp, synthetic construct
  source 1..35: mol_type = other DNA; note = synthetic promoter sequence
tttacggctagctcagccctaggtattatgctagc

SEQ ID NO: 22 - DNA, 675 bp, Saccharomyces cerevisiae
  source 1..675: mol_type = genomic DNA
cgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaa

SEQ ID NO: 23 - DNA, 405 bp, Saccharomyces cerevisiae
  source 1..405: mol_type = genomic DNA
caaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaaa